(19) United States
(12) Patent Application Publication
(10) Pub. No.: US 2026/0242348 A1
(43) Pub. Date: Aug. 20, 2026

(54) HIGHLY ATOM ECONOMICAL PROCESS FOR SYNTHESIS OF NOVEL CO-INITIATOR FOR PHOTO-INDUCED RADICAL POLYMERISATION

(71) Applicant: SHIV NADAR (Institution of Eminence Deemed To Be University), Nagar (IN)

(72) Inventors: Ajoy KAPAT, Nagar (IN); Asrar AHMAD, Nagar (IN); Garvisha MITTAL, Nagar (IN)

(73) Assignee: SHIV NADAR (Institution of Eminence Deemed To Be University), Nagar (IN)

(21) Appl. No.: 19/164,727

(22) PCT Filed: Jun. 26, 2023
(86) PCT No.: PCT/IN2023/050615

§ 371 Date: Sep. 12, 2025

(30) Foreign Application Priority Data
Mar. 13, 2023 (IN) 202311016662

(51) Int. Cl. C07D 327/06; C07D 317/54; C07D 321/10; C07D 327/04; C07D 339/06; C07D 411/06

(52) U.S. Cl. CPC C07D 327/06; C07D 317/54; C07D 321/10; C07D 327/04; C07D 339/06; C07D 411/06

(57) ABSTRACT

The present invention relates to a base catalyzed sequential conjugate addition reaction for the synthesis of a novel and efficient co-initiator for Photo Induced Radical Polymerization. The process comprises synthesis of new co-initiator having 6,5 and 6,6-bicyclic scaffolds as central core. The process comprises reacting thiocarboxylic acid, catechol, thiocatechol, benzodithiol, benzothiocatechol and binol with ketone substrates at room temperature with a base and a solvent to yield the novel co-initiator compound of Formula 3, Formula 5, Formula C and Formula Z.

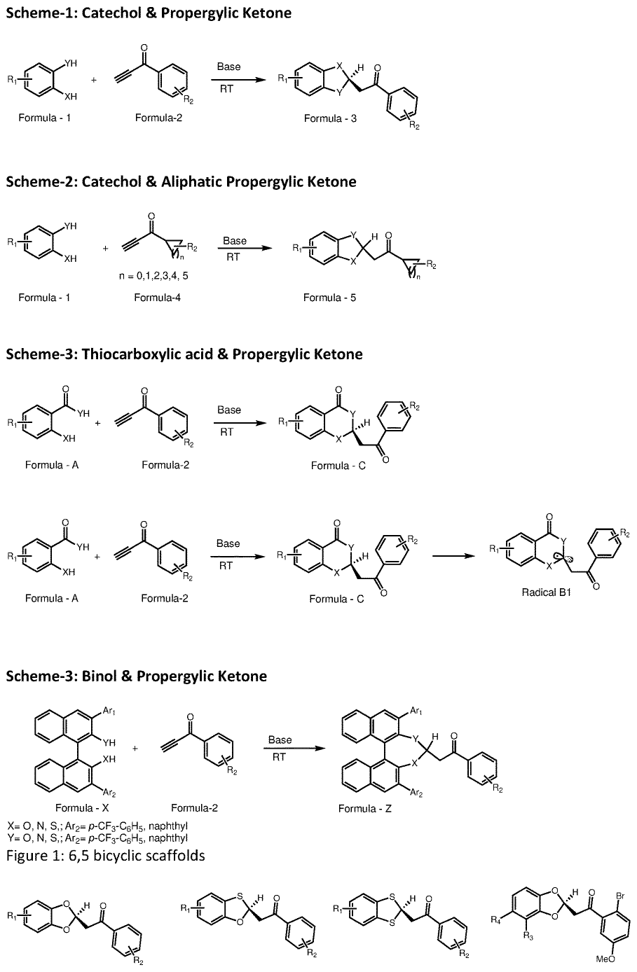

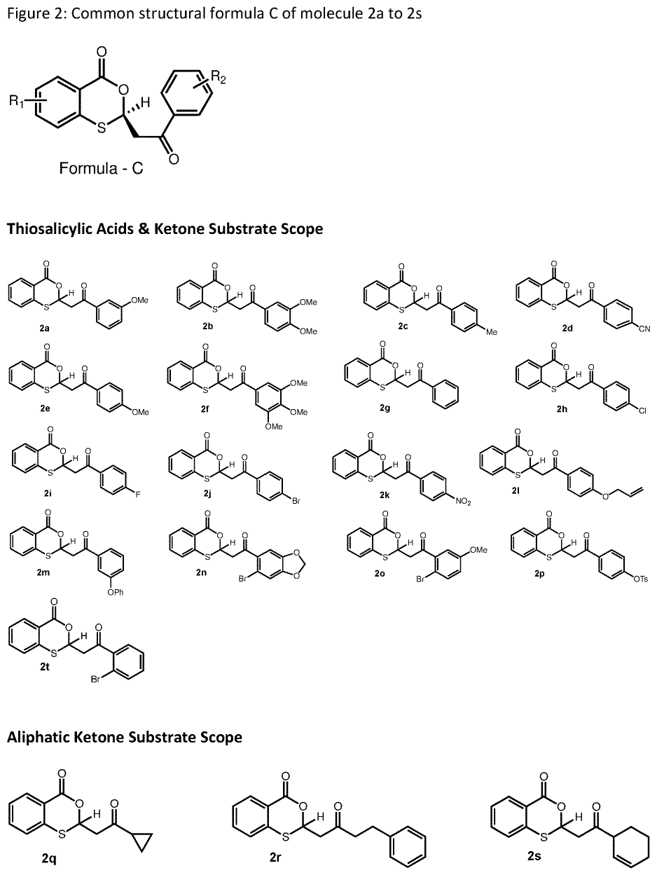

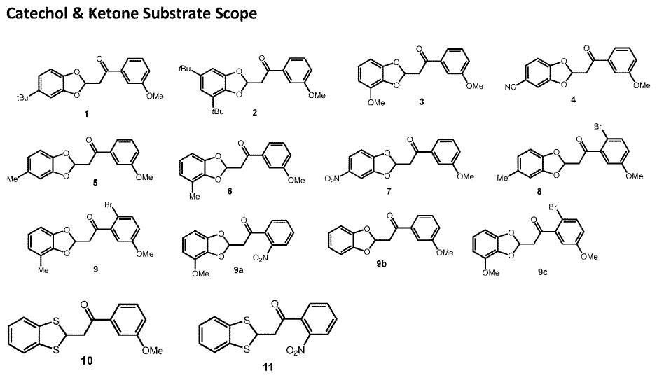

### FIELD OF THE INVENTION

The present invention relates to novel co-initiators for photo-induced radical polymerization reactions. Furthermore, the present invention relates to a process of base catalyzed sequential conjugate addition reaction for the synthesis of a novel and efficient co-initiator for photo induced radical polymerization.

### BACKGROUND OF THE INVENTION

Photo-induced radical polymerization reaction (PRP) is becoming very important tool in the area of 3D-printing, coating, dental restoration process, micro-electronics, photolithography, data storage application, etc. This radical polymerization process has many advantages which includes availability of LED light sources with wave length-flexibility, broad availability of the monomer formulation, solvent less method and requirement of less energy. These aspects are particularly very relevant under current scenario, as there is a need to synthesize polymeric materials in more sustainable way without comprising on material performance. Mechanistically photopolymerization proceeds via absorption of photon by the chromophore (initiator) and subsequently it generates radical, which initiate the polymerization process. This process can be classified into two classes based on the decomposition mechanism of the photo initiator, a type-I and type-II. In case of type-I, photo initiator undergoes alfa-fragmentation to generate radical, which initiate the polymerization reaction. Whereas, type-II photo initiator at first goes to its corresponding triplet state under photochemical conditions. Then this triplet species reacts with a suitable co-initiator to generate radical via electron/proton transfer (for tertiary amine) or via proton transfer (for cyclic ketal). This generated radical species react with the monomer molecules, which further undergoes polymerization reaction.

Due to the inherent limitation of type-I photo initiators (example—high-cost factor, oxidative quenching, etc.) type-IT photo initiators are used more commonly in PRP in various applications one of them being dental restoration. Typically, tertiary amine such as ethyl 4-(dimethylamino) benzoate (EDMAB) has been used extensively as co-initiator in the prior art for dental restoration application. In spite of its several advantages, amine mediated PRP reaction suffers from few drawbacks such as yellowing and toxicity which are becoming major concerns for the dental patients. Mechanistically, formation of nitrogen centered radical undergoes aerial oxidation to form the N-oxides, which is the responsible factor for yellowing. To avoid these limitations of the hazardous tertiary amine co-initiator, significant amount of research activity has been dedicated to address these challenges.

Further, some important prior art in the area of PRP reaction are discussed briefly below.

Sajjad et al., authored photoinduced electron transfer reactions for macromolecular synthesis review paper provides a comprehensive account of the fundamentals and applications of photoinduced electron transfer reactions in polymer synthesis. Besides traditional photopolymerization methods, namely free radical and cationic polymerizations, step-growth polymerizations involving electron transfer processes are included.

Aydogan, C.; Yilmaz, G.; Shegiwal, A.; Haddleton, D. M.; Yagci, Y. Photoinduced Controlled/Living Polymerizations. Angew. Chem. Int. Ed. Engl. 2022, a review article that disclosed strategies for photoinduced controlled living radical and cationic polymerization and their combinations for the fabrication of different macromolecular architectures. The wavelength tunability and the advantages of the light-induced reactions carried out under mild conditions are presented. Also disclosed are discussion of mechanistic details highlights differences as well as parallels between different systems for different polymerization methods and monomer applicability.

Joseph B. Nganga, Young Jae Jung, Yunpei Si, Minyoung Kim, Hyebin Ko, Gil Tae Hwang, Hye Jin Lee, Hong-In Lee, Jungkyu K. Lee, Photoinduced radical polymerization by methyl fluoresceins under visible light and the application to signal amplification of hydrogen peroxide, Dyes and Pigments, Volume 200, 2022, disclosed visible-light-induced radical polymerization initiated by methyl fluorescein and its derivatives (e.g., methyl ether of fluorescein, methyl ether ester of fluorescein, and methyl ester of fluorescein), which act as photoredox catalysts. The structural alterations of fluorescein noticeably affect their photoredox catalytic performance, which increases in the following order: methyl ether of fluorescein<methyl ether ester of fluorescein<methyl ester of fluorescein≤fluorescein. Particularly, the methylation on the phenolic hydroxyl group reduces their performance mainly due to the restraint of tautomerization. Based on this systematic investigation, methyl ether fluorescein bearing aryl boronate as an H₂O₂-selective photoredox catalyst has been synthesized, and use it to amplify the signal of H₂O₂ through the photoinduced radical polymerization. In the presence of H₂O₂, this photoredox catalyst successfully initiates the polymerization of poly(ethylene glycol) diacrylate and N-vinylpyrrolidone, forming a polymeric hydrogel in 1 min under visible-light irradiation.

To avoid limitations of the hazardous tertiary amine co-initiator, significant amount of research activity has been dedicated to address these challenges. Benzodioxole derivatives (BDO) turned out to be suitable alternative for dental application. More co-initiator loading and delayed polymerization time are the major drawbacks for BDO-CQ combination. In addition to that certain amount of yellowing is also observed in this case. The present invention was conceived to overcome, the drawbacks of BDO-CQ co-initiators, particularly for dental restoration application.

To avoid current limitation of BDO based co-initiators and to improve the efficiency of the type-II photoinitiator mediated PRP reaction, the present invention discloses new co-initiator having 6,5 and 6,6-bicyclic scaffolds A & B as central core. Several methodologies were reported in the literature for the synthesis of benzoxathiinones and benzodioxinones derivatives having quaternary centre next to the heteroatoms. In background art is disclosed, Synthesis of benzodioxole, benzodithiole, benzothioxole, 3,1-benzoxathiin-4-ones and benzodioxine derivatives having a labile hydrogen next to the heteroatoms is relatively less explored. However, these reported protocols suffer from the few drawbacks such as limited substrate scope, low yielding and requirement of high temperature.

Tripathi, V. K.; Venkataramani, P. S.; Mehta, G. Addition of nitrogen-, oxygen-, and sulphur-containing nucleophiles to aryl ethynyl ketones, Journal of Chemical Society, Perkin Transactions, Issue 0, 1979 disclosed, addition of some nitrogen-, oxygen-, and sulphur-containing nucleophiles to the aryl ethynyl ketones has been examined. Addition of o-phenylenediamine and o-aminophenol yielded the corresponding cis-β-aminovinyl ketones. Addition of secondary amines (morpholine and piperidine) yielded the trans-β-aminovinyl ketones. Catechol reacted with aryl ethynyl ketones to furnish 1,3-benzodioxole derivatives, salicylic acid and 2-hydroxy-3-naphthoic acid to give the oxalactones. However, 2-hydroxy-1-naphthoic acid yielded a mixture of the oxalactones and 3-naphthoxy-1-arylprop-2-enones. Similarly, thiosalicylic acid gave the corresponding thialactones as addition products. Salicylaldehyde reacted with ethynyl phenyl ketone to give 3-benzoylchrom-3-en-2-ol.

In various embodiments of the present invention is disclosed facile synthetic methodology utilizing inexpensive and non-hazardous, environmentally benign and naturally abundant materials for high-yielding reactions to produce highly thermally stable small molecules, which is easily processable and promote catalytic photo-induced radical polymerization reaction at room temperature with very high efficiency.

### OBJECTIVES OF THE INVENTION

The principal objective of the present invention is to provide a process for synthesis of novel co-initiator for the photo-induced radical polymerization reaction.

It is another object of the present invention to provide novel co-initiators for the photo-induced radical polymerization reaction that has the potential to overcome drawbacks of amine mediated PRP reaction like yellowing and toxicity. Another objective of the present invention is to provide a process for synthesis of new co-initiator having 6, 5, 6, 6-bicyclic and 6,7,6 scaffolds as central core.

It is yet another objective of the present invention to provide process of base catalyzed sequential conjugate addition reaction which provides very efficient access of substituted [1,3]-benzodioxole, [1,3]-benzodithianes, [1,3]-benzothioxole, [1,3]-benzoxathiin-4-ones and [1,3]dioxepin derivative.

It is another object of the present invention to provide substituted [1,3]-benzodioxole, [1,3]-benzodithianes, [1,3]-benzothioxole, [1, 3]-benzoxathiin-4-ones and [1,3]dioxepin derivative with very high yield, high atom-economy and broad ranges of substrate scope at room temperature.

It is another object of the present invention to provide substituted [1,3]-benzodioxole, [1,3]-benzodithianes, [1,3]-benzothioxole, 1, 3-benzoxathiin-4-ones and [13]dioxepin derivative in gram scale.

### SUMMARY OF THE INVENTION

Accordingly, the present invention relates to a process for the synthesis of novel co-initiators for the photo-induced radical polymerization reaction and the reaction products thereof.

In one aspect of the present invention is provided, a process of preparation of co-initiator for photo induced radical polymerization, comprising steps:
i. reacting compound of Formula 1 with compound of formula 2, in the presence of a base at room temperature yields compound of formula 3

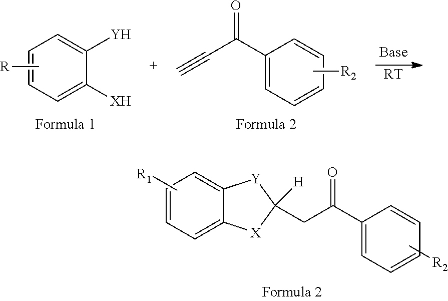

ii. reacting compound of Formula 1 with compound of formula 4, in presence of a base at room temperature yields compound of formula 5;

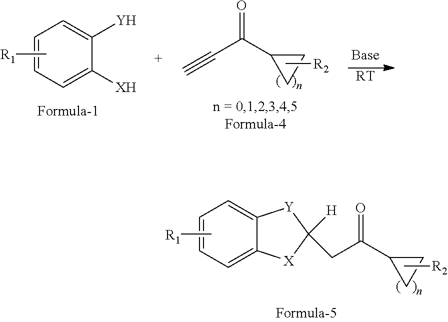

wherein, X is selected from the group comprising of O, S, N, Se and Te; Y is selected from the group comprising of O, S, N, Se and Te;
wherein R₁ is selected from the group consisting Hydro, alkyl, branched chain alkyl, aryl, t-butyl, methyl, cyano, o-methyl, Nitro and also comprises tert-Butyloxycarbonyl (BocHN); and
various acyclic and cyclic ketones in formula 5 are selected from n=0, 1, 2, 3, 4, 5
R₂ is selected from group comprising of OMe, Me, CN, Methyl, Cl, F, Br, NO₂, OPh, OTs, allylic and the like.
iii. reacting compound of Formula A with compound of formula 2 (ketone), in the presence of a base at room temperature yields compound of formula C;

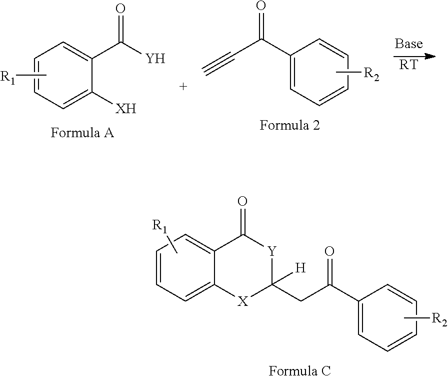

wherein X is selected from O, S, N, Se and Te;
Y is selected from O, S, N, Se and Te;
wherein R₁ is selected from the group consisting Hydro, alkyl, branched chain alkyl, aryl, t-butyl, methyl, cyano, o-methyl, Nitro and also comprises tert-Butyloxycarbonyl (BocHN); and
R₂ is selected from group comprising of OMe, Me, CN, Methyl, Cl, F, Br, NO₂, OPh, OTs, allylic and the like.
iv. reacting compound of Formula X with compound of formula 2, in the presence of a base at room temperature yields compound of formula Z

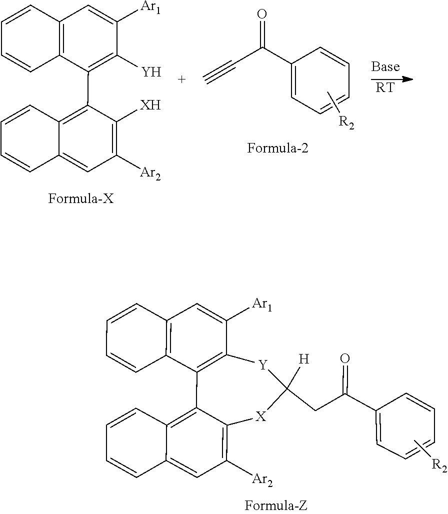

wherein,
   X=O, N, S
   Y=O, N, S
   Ar₁=p-CF₃—C₆H₅, naphthyl
   Ar₂=p-CF₃—C₆H₅, naphthyl
wherein, R₂ is selected from group comprising of OMe, Me, CN, Methyl, Cl, F, Br, NO₂, OPh, OTs, allylic and the like.

In one aspect of the present invention, the process of preparation of co-initiator for photo induced radical polymerization as claimed in claim 1, wherein the ratio of compound of Formula A, Formula 1 and Formula X with Formula 2 is in the ratio of 1:1 equivalents and Formula 1 with Formula 4 is in a ratio of 1:1 equivalents.

In one aspect of the present invention, the process of preparation of co-initiator for photo induced radical polymerization, in which the base is selected from potassium carbonate, sodium carbonate, sodium hydroxide, sodium acetate, potassium tertiary butoxide, sodium tertiary butoxide, barium hydroxide, sodium amide in a concentration range of 2-10 mol %.

In exemplary embodiments of the present invention using K₂CO₃ or potassium carbonate as catalyst turned to be the best catalyst.

In another aspect of the present invention, the process of preparation of co-initiator for photo induced radical polymerization, in which the solvent is selected from polar and non-polar solvents such as dichloromethane (DCM), methanol, acetonitrile, toluene, tetrahydrofuran.

In provided exemplary embodiments of the present invention and as provided in the process schemes of and the present invention, dichloromethane (DCM) proves to be the best solvent, in a concentration range of 2-10 ml.

In another aspect of the present invention, the process of preparation of co-initiator for photo induced radical polymerization in which the reaction time to attain compound of Formula-3 is 5 min to 5.5 hrs.

In another aspect of the present invention, the process of preparation of co-initiator for photo induced radical polymerization in which the reaction time to attain compound of Formula-5 is up to 5.5 hrs.

In yet another aspect of the present invention, the process of preparation of co-initiator for photo induced radical polymerization in which the reaction time to attain compound of Formula-C is 5.5 hrs.

In yet another aspect of the present invention, the process of preparation of co-initiator for photo induced radical polymerization in which the reaction time to attain compound of Formula-Z is 5.5 hrs.

In yet another aspect of the present invention, the process of preparation of co-initiator for photo induced radical polymerization in which the Formula 3 compound in scheme (i) comprises:

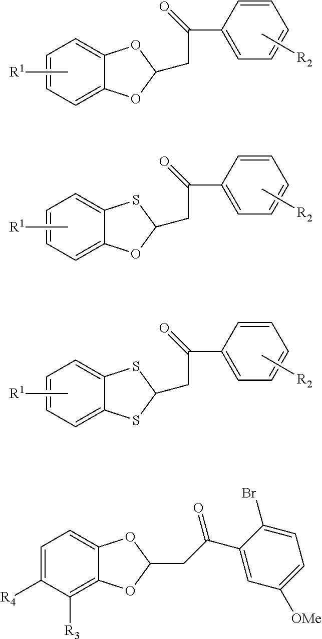

wherein,
R₁ is selected from group comprising of Hydro, alkyl, branched chain alkyl, aryl, t-butyl, methyl, cyano, o-methyl, nitro and also comprises tert-Butyloxycarbonyl (BocHN);
R₂ is selected from group comprising of OMe, Me, CN, Methyl, Cl, F, Br, NO₂, OPh, OTs, allylic and the like.
R₃ is selected from the group comprising of Me, H, and
R₄ is selected from group comprising of Me, H.

In further aspect of the present invention, the process of preparation of co-initiator for photo induced radical polymerization in which the formula-C is the compound comprising:

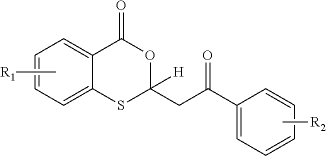

wherein, R₁ is selected from group comprising of Hydro alkyl, branched chain alkyl, aryl, t-butyl, methyl, cyano, o-methyl, nitro and also comprises tert-butyloxycarbonyl (BocHN);
R₂ is selected from group comprising of OMe, Me, CN, Methyl, Cl, F, Br, NO₂, OPh, OTs, allylic and the like

In further aspect of the present invention, the compounds of Formula 3, Formula 5, Formula C and Formula Z are selected from
1. 2-(2-(3-methoxyphenyl)-2-oxoethyl)-4H-benzo[d][1,3]oxathiin-4-one (2a)
2. 2-(2-(3,4-dimethoxyphenyl)-2-oxoethyl)-4H-benzo[d][1,3]oxathiin-4-one (2b)
3. 2-(2-oxo-2-(p-tolyl)ethyl)-4H-benzo[d][1,3]oxathiin-4-one (2c)
4. 4-(2-(4-oxo-4H-benzo[d][1,3]oxathiin-2-yl)acetyl)benzonitrile (2d)
5. 2-(2-(4-methoxyphenyl)-2-oxoethyl)-4H-benzo[d][1,3]oxathiin-4-one (2e)
6. 2-(2-oxo-2-(3,4,5-trimethoxyphenyl)ethyl)-4H-benzo[d][1,3]oxathiin-4-one (2f)
7. 2-(2-oxo-2-phenylethyl)-4H-benzo[d][1,3]oxathiin-4-one (2g)
8. 2-(2-(4-chlorophenyl)-2-oxoethyl)-4H-benzo[d][1,3]oxathiin-4-one (2h)
9. 2-(2-(4-fluorophenyl)-2-oxoethyl)-4H-benzo[d][1,3]oxathiin-4-one (2i)
10. 2-(2-(4-bromophenyl)-2-oxoethyl)-4H-benzo[d][1,3]oxathiin-4-one (2j)
11. 2-(2-(4-nitrophenyl)-2-oxoethyl)-4H-benzo[d][1,3]oxathiin-4-one (2k)
12. 2-(2-(4-(allyloxy)phenyl)-2-oxoethyl)-4H-benzo[d][1,3]oxathiin-4-one (2l)
13. 2-(2-oxo-2-(3-phenoxyphenyl)ethyl)-4H-benzo[d][1,3]oxathiin-4-one (2m)
14. 2-(2-(6-bromobenzo[d][1,3]dioxol-5-yl)-2-oxoethyl)4Hbenzo[d][1,3]oxathiin-4-one (2n)
15. 2-(2-(2-bromo-5-methoxyphenyl)-2-oxoethyl)-4H-benzo[d][1,3]oxathiin-4-one (2o)
16. 4-(2-(4-oxo-4H-benzo[d][1,3]oxathiin-2-yl)acetyl)phenyl4-methyl benzenesulfonate (2p)
17. 2-(2-cyclopropyl-2-oxoethyl)-4H-benzo[d][1,3]oxathiin-4-one (2q)
18. 2-(2-oxo-4-phenylbutyl)-4H-benzo[d][1,3]oxathiin-4-one (2r)
19. 2-(2-(cyclohex-2-en-1-yl)-2-oxoethyl)-4H-benzo[d][1,3]oxathiin-4-one (2s)
20. 2-(2-(2-bromo-5-methoxyphenyl)-2-oxoethyl)-4H-benzo[d][1, 3]oxathiin-4-one (2t)
21. 2-(5-(tert-butyl)benzo[d][1,3]dioxol-2-yl)-1-(3-methoxyphenyl)ethan-1-one (1)
22. 2-(4,6-di-tert-butyl benzo[d][1,3]dioxol-2-yl)-1-(3 methoxyphenyl)ethan-1-one (2)
23. 2-(4-methoxybenzo[d][1,3]dioxol-2-yl)-1-(3-methoxyphenyl)ethan-1-one (3)
24. 2-(2-(3-methoxyphenyl)-2-oxoethyl) benzo[d][1,3]dioxole-5-carbonitrile (4)
25. 1-(3-methoxyphenyl)-2-(5-methylbenzo[d][1,3]dioxol-2-yl)ethan-1-one (5)
26. 1-(3-methoxyphenyl)-2-(4-methylbenzo[d][1,3]dioxol-2-yl)ethan-1-one (6)
27. 1-(3-methoxyphenyl)-2-(5-nitrobenzo[d][1,3]dioxol-2-yl)ethan-1-one (7)
28. 1-(2-bromo-5-methoxyphenyl)-2-(5-methylbenzo[d][1,3]dioxol-2-yl)ethan-1-one (8)
29. 1-(2-bromo-5-methoxyphenyl)-2-(4-methoxybenzo[d][1,3]dioxol-2-yl)ethan-1-one (9c)
30. 2-(4-methoxybenzo[d][1,3]dioxol-2-yl)-1-(2-nitrophenyl) ethan-1-one (9a)
31. 2-(benzo[d][1,3]dioxol-2-yl)-1-(3-methoxyphenyl)ethan-1-one (9b)
32. 2-(benzo[d][1,3]oxathiol-2-yl)-1-(3-methoxyphenyl) ethan-1-one (10)
33. 2-(benzo[d][1,3]dithiol-2-yl)-1-(2-nitrophenyl)ethan-1-one (11)
34. 2-(benzo[d][1,3]dithiol-2-yl)-1-(3-methoxyphenyl) ethan-1-one (12)
35. 2-(dinaphtho[2,1-d:1′,2′-f][1,3]dioxepin-4-yl)-1-(3 methoxyphenyl)ethan-1-one (13)

### DETAILED DESCRIPTION OF THE INVENTION

The present invention as embodied by “HIGHLY ATOM ECONOMICAL PROCESS FOR SYNTHESIS OF NOVEL CO-INITIATOR FOR PHOTO-INDUCED RADICAL POLYMERISATION”, succinctly fulfils the above-mentioned need(s) in the art. The present invention has objective(s) arising as a result of the above-mentioned need(s), said objective(s) being enumerated below. In as much as the objective(s) of the present invention are enumerated, it will be obvious to a person skilled in the art that the enumerated objective(s) are not exhaustive of the present invention in its entirety, and are enclosed solely for the purpose of illustration. Further, the present invention encloses within its scope and purview, any structural alternative(s) and/or any functional equivalent(s) even though such structural alternative(s) and/or any functional equivalent(s) are not mentioned explicitly herein or elsewhere, in the present disclosure. The present invention therefore encompasses also, any improvisation(s)/modification(s) applied to the structural alternative(s)/functional alternative(s) within its scope and purview. The present invention may be embodied in other specific form(s) without departing from the spirit or essential attributes thereof.

Throughout this specification, the use of the word “comprises” and variations such as “comprises” and “comprising” may imply the inclusion of an element or elements not specifically recited.

The present invention in a principal embodiment discloses a process of synthesis novel co-initiator for photo induced radical polymerization reaction. The invention was conceived to overcome the drawbacks in the [1, 3]-Benzodioxole or BDO based co-initiators in certain applications due to quick oxidation. The synthetic scheme of the present invention resulted in reaction products, comprising 6, 5 (Compounds of Formula 3 in Scheme 1) and 6, 6 scaffold (Compounds of Formula C) based compounds which are novel co-initiators for photoinduced radical polymerization reaction.

From the mechanistic point of view BDO derivatives act as a hydrogen atom donor in the PRP reaction. According to the present invention, it was anticipated that resultant radicals A1 (Scheme 1) & B1 would be more stable due to the presence of captodative stabilization effect. Hence, bond dissociation energy (BDE) would be less and as a result these scaffolds (Formula 3 and Formula C) would be more efficient co-initiator for type-II photoinitiator mediated PRP reaction. The present invention developed efficient synthetic strategy to have easy access of these bicyclic scaffolds. Retro-synthetically speaking nucleophilic addition of suitable substrate having two nucleophilic centres to the propargylic ketone would lead to the formation of the desired skeleton via sequential conjugate addition reaction.

The various embodiments of the present invention are discussed in detail below.

In one embodiment is disclosed a process for synthesis of the novel co-initiator comprising steps;

Firstly, a compound of Formula 1 is reacted with a substrate, i.e., propargyl ketone (Formula 2) to result in a novel co-initiator of Formula 3 as illustrated in Scheme 1.

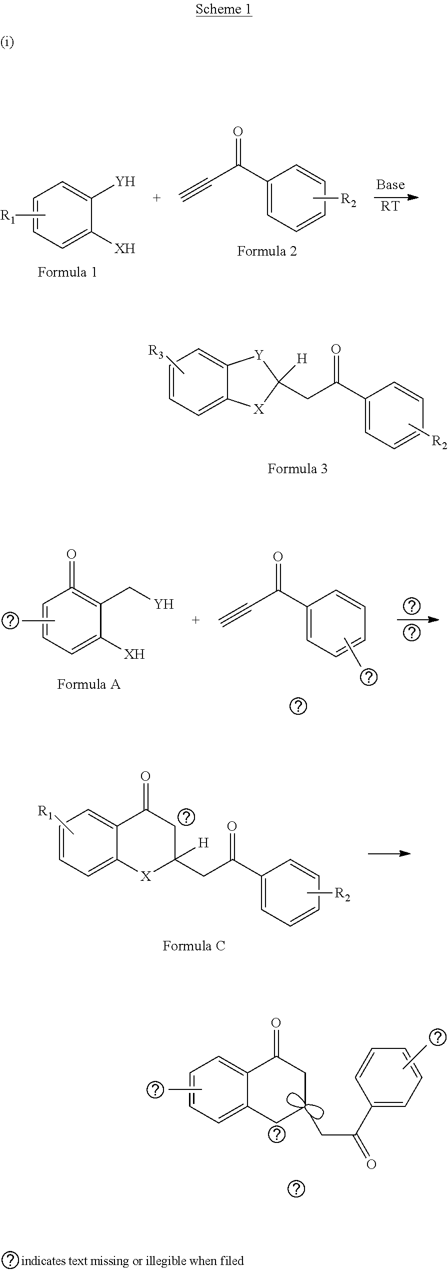

(ii) a compound of Formula 1 is reacted with a substrate, i.e., propargyl ketone (Formula 4), in a ratio of 1:1 equivalents, in the presence of base and at room temperature, results in a novel co-initiator of Formula 5, as illustrated in Scheme 1.

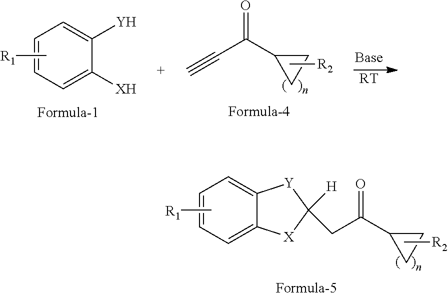

As provided in Scheme 1, Formula 1 may comprise,
X=O, S, N, Se, and Te,
Y=O, S, N, Se, and Te.

In Formula 2 and Formula 3 i.e. propargyl ketone,
R₁ is selected from group comprising of Hydro, alkyl, branched chain alkyl, aryl, t-butyl, methyl, cyano, o-methyl, Nitro and also comprises tert-Butyloxycarbonyl (BocHN);
various acyclic and cyclic ketones of Formula 4 is selected from n=0, 1, 2, 3, 4
R₂ is selected from group comprising of OMe, Me, CN, Methyl, Cl, F, Br, NO₂, OPh, OTs, allylic and the like.

In a preferred embodiment of the present invention compound of Formula 1 is selected from
X=O, Y=O Catechol
X=O, Y=S Thiocatechol;
X=S, Y=S Dithiocatechol

The reaction proceeds as weighed quantity of compound of formula 1 (Reactant) is reacted with a ketone, preferably propargyl ketone (Formula 2) (1:1 equivalents by weight) in the presence of catalytic amount of base (2-10 mol %) at room temperature (RT) and for a time period of 5 minutes to 5.5 hours depending on the reactant. The crude material was further purified by methods such as short plug of silica, recrystallization, column chromatography, silica gel and column chromatography.

The base catalyst is selected from potassium carbonate (K₂CO₃), sodium hydroxide (NaOH), sodium acetate (NaOAc), potassium tertiarybutoxide (KOtBu), sodium tertiarybutoxide (NaOtBu), barium hydroxide (Ba(OH)₂), sodium amide (NaNH₂), sodium carbonate (Na₂CO₃), and potassium carbonate turned to be the best catalyst, also the temperature sustained in room temperature (RT), which is one of the novel aspect of the present invention. The base is in the mol % of 2-10.

Scheme 1 depicts the process for synthesis of 6,5 scaffold structure compounds of the Novel co-initiator. In exemplary embodiments the propargyl ketone is represented by the compound of Formula 3, potassium carbonate (K₂CO₃), is the base catalyst and reactant is compound of formula 3 selected from catechol or thiocatechol.

Representative compounds of Scheme 1 are provided below:
1. The below compounds are representative of compounds of Formula 3, wherein X=O, Y=O and the reactant i.e. compound of Formula 1 is Catechol.

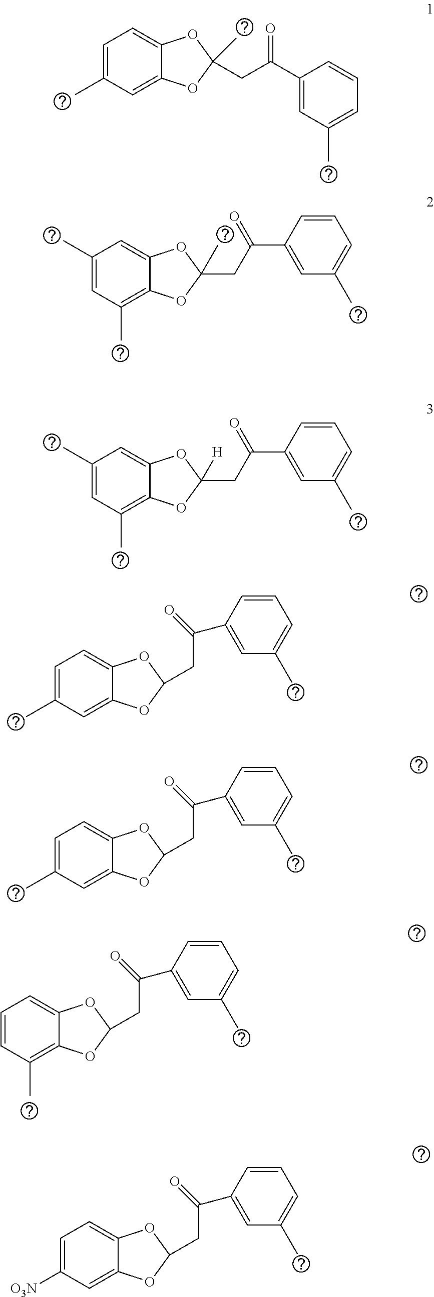

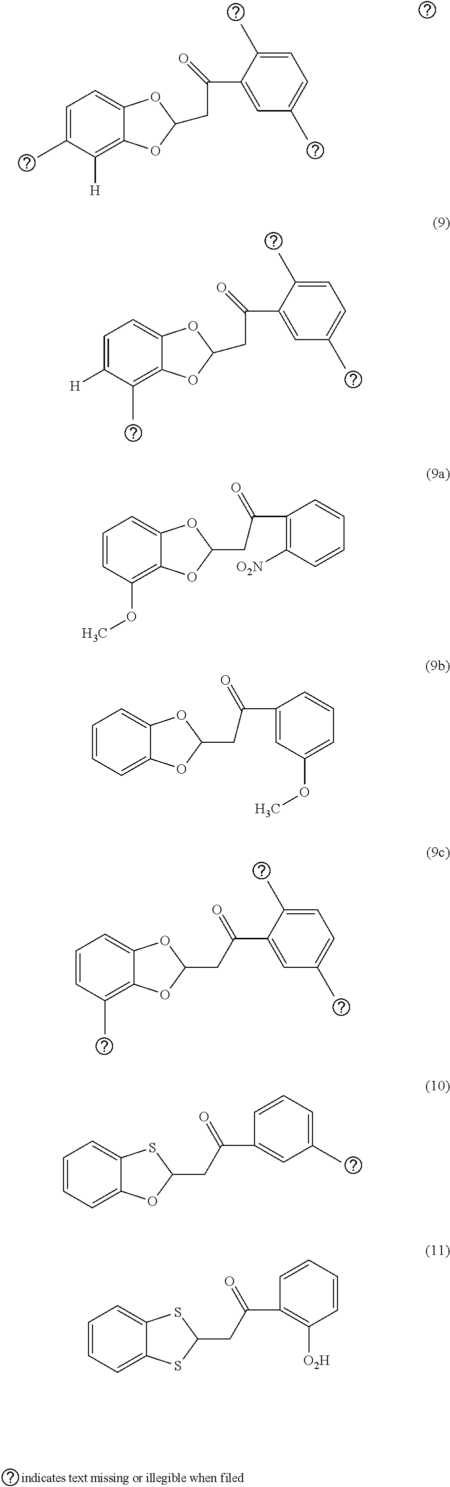

2. Representative compounds (10, 11) of Formula 3, wherein X=S, Y=O and the reactant i.e., compound of Formula 1 is thiocatechol.

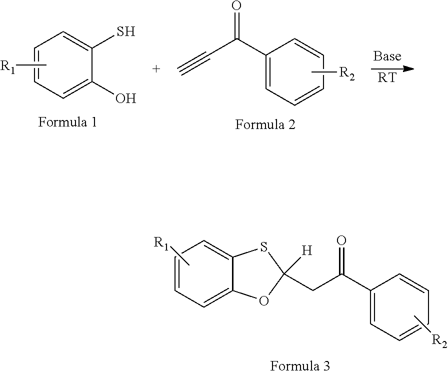

3. Representative compound (12) of Formula 3, wherein X=S, Y=S and the reactant i.e., compound of Formula 1 is dithiocatechol.

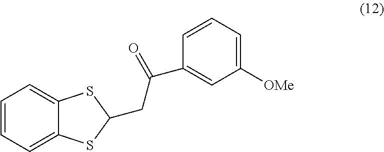 [SMILES: COc1cccc(C(=O)CC2Sc3ccccc3S2)c1]

In another preferred embodiment is provided a process for synthesis of a 6, 6 scaffold structure compounds of general Formula C as represented in Scheme 2

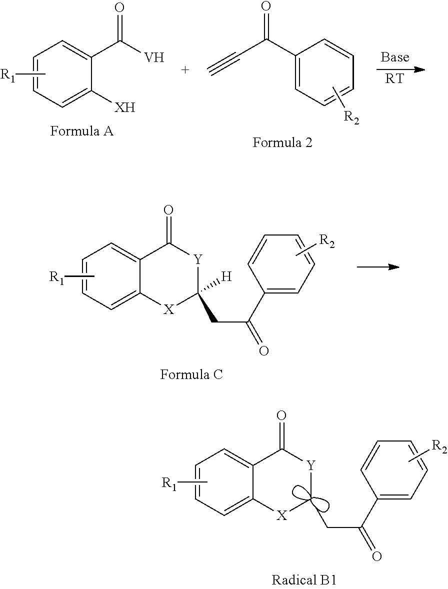

wherein:
X is selected from O, S, N, Se and Te;
Y is selected from O, S, N, Se and Te;
R₁ is selected from group comprising of Hydro, alkyl, branched chain alkyl, aryl, t-butyl, methyl, cyano, o-methyl, Nitro and also comprises tert-bButyloxycarbonyl (BocHN);
R₂ is selected from group comprising of OMe, Me, CN, Methyl, Cl, F, Br, NO₂, OPh, OTs, allylic and the like.

The reaction proceeds as weighed quantity of compound of formula A (Reactant) which is a thiocarboxylic acid is reacted with a ketone, preferably propargyl ketone (Formula 2), in a ratio of 1:1 equivalents, in the presence of a base (2-10 mol %) at room temperature (RT) and for a time period of 5 minutes to 5.5 hours depending on the reactant. The crude material was further purified by methods such as short plug of silica, recrystallization, column chromatography, silica gel and column chromatography.

Also the base catalyst is selected from potassium carbonate K₂CO₃, sodium hydroxide (NaOH), sodium acetate (NaOAc), potassium tertiarybutoxide (KOtBu), sodium tertiarybutoxide (NaOtBu), barium hydroxide (Ba(OH)₂), sodium amide (NaNH₂), sodium carbonate (Na₂CO₃), and potassium carbonate turned to be the best catalyst also the temperature sustained in Room Temperature (RT).

In exemplary embodiment the base catalyst is selected from potassium carbonate K₂CO₃ (5 mol %) In a preferred embodiment of the present invention the compound of Formula A selected from

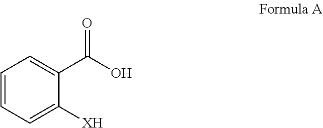

X=O, Salicylic acid
X=S, Thiosalicylic acid

In the preferred embodiment, the Compound of Formula A is the reactant which is selected from either thiosalicylic acid or salicylic acid, of Formula A

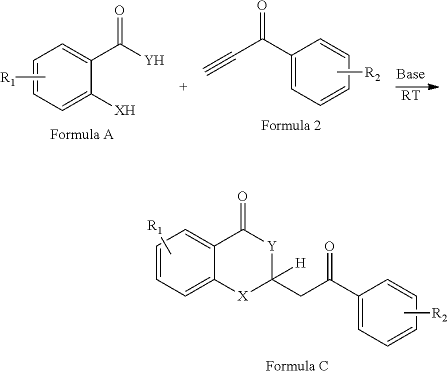

Representative compounds of Scheme 2 are provided below:
a. X=S, Y=O
   a. Ketone Substrate

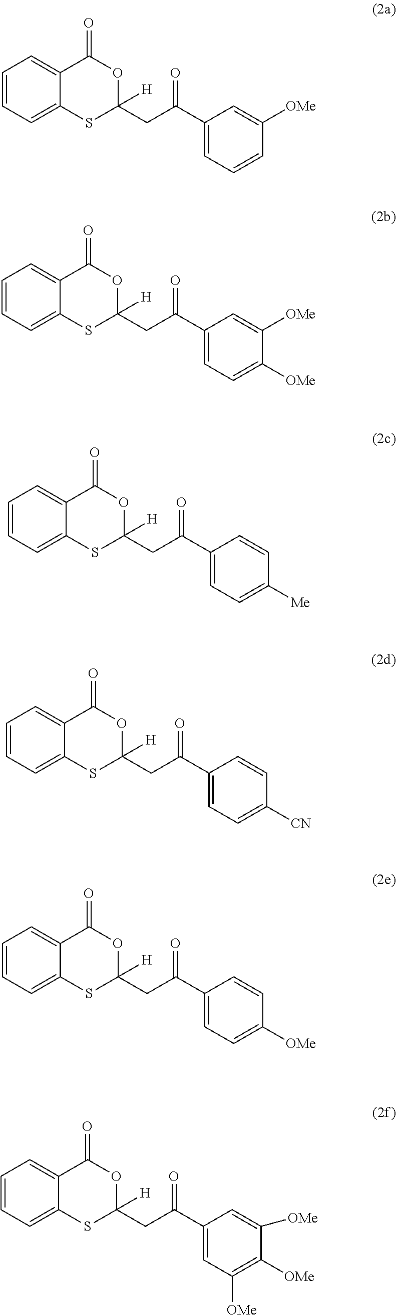 [SMILES: COc1cccc(C(=O)CC2OC(=O)c3ccccc3S2)c1 ; COc1ccc(C(=O)CC2OC(=O)c3ccccc3S2)cc1OC ; Cc1ccc(C(=O)CC2OC(=O)c3ccccc3S2)cc1 ; N#Cc1ccc(C(=O)CC2OC(=O)c3ccccc3S2)cc1 ; COc1ccc(C(=O)CC2OC(=O)c3ccccc3S2)cc1 ; COc1cc(C(=O)CC2OC(=O)c3ccccc3S2)cc(OC)c1OC]

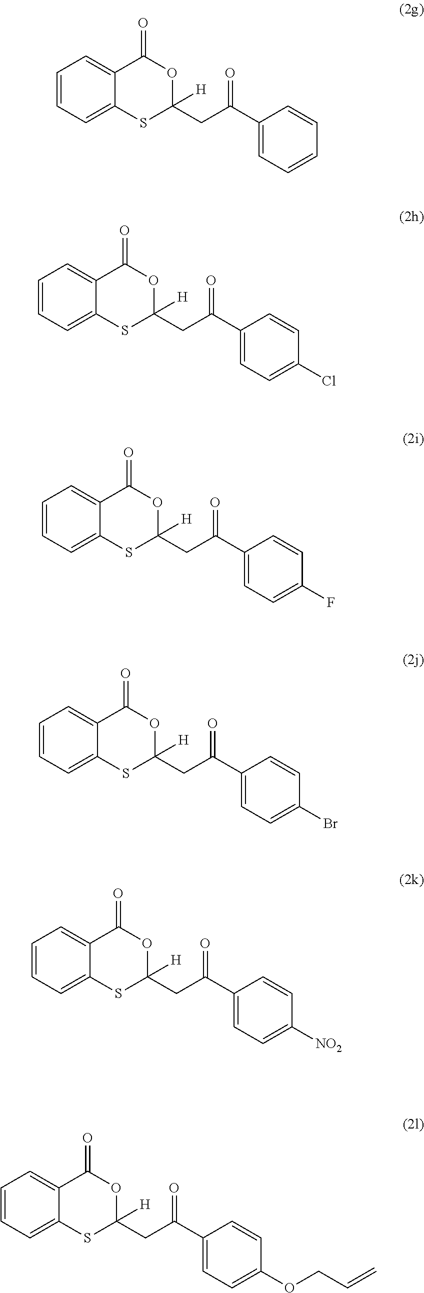 [SMILES: O=C(CC1OC(=O)c2ccccc2S1)c1ccccc1 ; O=C(CC1OC(=O)c2ccccc2S1)c1ccc(Cl)cc1 ; O=C(CC1OC(=O)c2ccccc2S1)c1ccc(F)cc1 ; O=C(CC1OC(=O)c2ccccc2S1)c1ccc(Br)cc1 ; O=C(CC1OC(=O)c2ccccc2S1)c1ccc([N+](=O)[O-])cc1 ; C=CCOc1ccc(C(=O)CC2OC(=O)c3ccccc3S2)cc1]

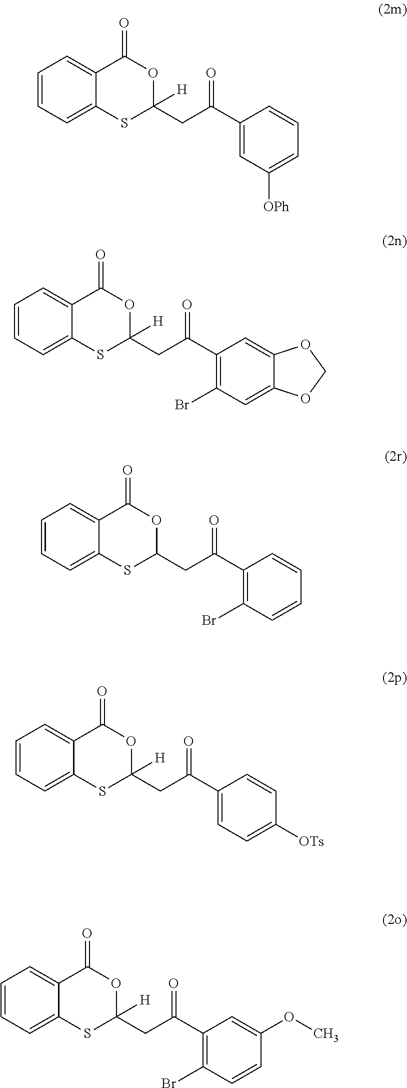 [SMILES: O=C(CC1OC(=O)c2ccccc2S1)c1cccc(Oc2ccccc2)c1 ; O=C(CC1OC(=O)c2ccccc2S1)c1cc2c(cc1Br)OCO2 ; O=C(CC1OC(=O)c2ccccc2S1)c1ccccc1Br ; Cc1ccc(S(=O)(=O)Oc2ccc(C(=O)CC3OC(=O)c4ccccc4S3)cc2)cc1 ; COc1ccc(Br)c(C(=O)CC2OC(=O)c3ccccc3S2)c1]

### 2. Aliphatic Ketone Substrate

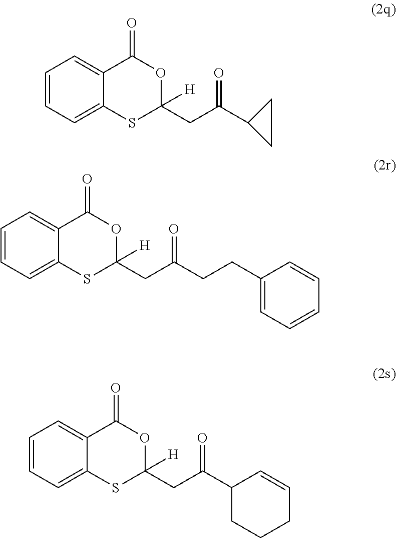 [SMILES: O=C1OC(CC(=O)C2CC2)Sc2ccccc21 ; O=C(CCc1ccccc1)CC1OC(=O)c2ccccc2S1 ; O=C1OC(CC(=O)C2C=CCCC2)Sc2ccccc21]

### Scheme 3

### BINOL Derivatives

In scheme 3, a compound of Formula X is reacted with ketone (propargyl ketone) of formula 2, in a ratio of 1:1 equivalents, at RT in the presence of a base (2-10 mol %) to yield compound of Formula Z;

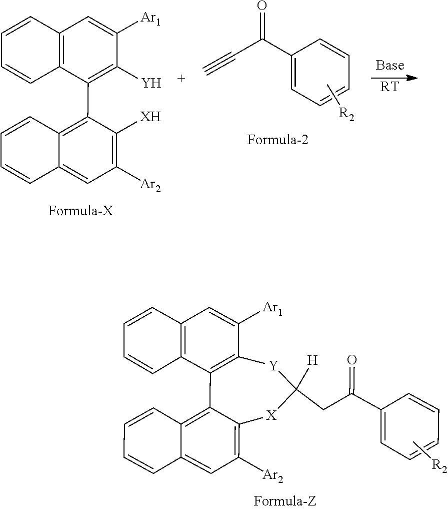

wherein,
X=O, N, S
Y=O, N, S
Ar₁=p-CF₃—C₆H₅, naphthyl
Ar₂=p-CF₃—C₆H₅, naphthyl
wherein, R₂ is selected from R₂ is selected from group comprising of OMe, Me, CN, Methyl, Cl, F, Br, NO₂, OPh, OTs, allylic and the like.

In one aspect of the present invention, the process of preparation of co-initiator for photo induced radical polymerization, in which the base is selected from potassium carbonate, sodium carbonate, sodium hydroxide, sodium acetate, potassium tertiary butoxide, sodium tertiary butoxide, barium hydroxide, sodium amide.

In exemplary embodiments of the present invention using K₂CO₃ or potassium carbonate as catalyst turned to be the best catalyst when used at a concentration of 5 mol %.

Representative compound of Formula Z is

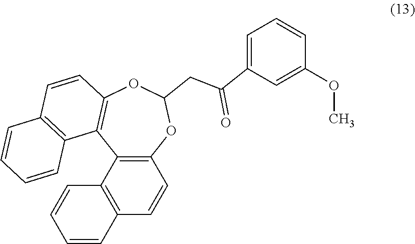 [SMILES: COc1cccc(C(=O)CC2Oc3ccc4ccccc4c3-c3c(ccc4ccccc34)O2)c1]

### WORKING EXAMPLES

The method comprises the steps of: The reactants for the Scheme 2 comprises: catechol, thiocatechol, di-thiocatechol and racemic BINOL, the sequential conjugate addition reaction conditions were initially optimized using the model substrates thiosalicylic acid (i) and 3-methoxyphenyl propargylic ketone (ii), under optimized reaction conditions, and the isolated yield following column chromatography was 72%.

Compound of Formula (iii) below is a representative compound of the 6,6 scaffolds where the reactant is a compound of Formula A which is a thiocarboxylic acid, particularly Thiocatechol.

The reaction as follows:

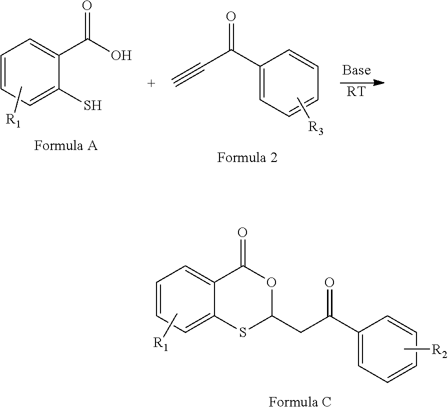

Formula A is Thiocarboxylic acid when reacted with substituted propargyl ketone (Formula 2) in the same reaction conditions as discussed under Scheme 2 (presence of base and at RT) results in a compound of Formula C, herein represented by formula C.

The principle of the present invention is to synthesize a library of molecules in which electron-donating and electron with drawing substituents included in aromatic rings.

A compound of Formula (C) synthesized using few of the reactants to attain 6-6 scaffold reaction a specific methodology is followed a respective ketone was added to a 25 mL round bottom flask equipped with a magnetic stir bar and was dissolved in DCM (4 ml), then K₂CO₃ (5 mol %) and thiosalicylic acid were added to it. The reaction mixture was stirred at room temperature for 5.5 hours, and the reaction's progress was monitored using TLC. DCM was removed under reduced pressure after the starting material had been consumed. To obtain the desired conjugate addition product, the crude material was purified using either flash silica gel column chromatography (20% EtOAc in Hexane) or recrystallization.

### Example 1

### (2a). Synthesis of 2-(2-(3-methoxyphenyl)-2-oxoethyl)-4H-benzo[d][1,3]oxathiin-4-one

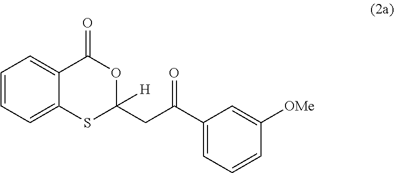 [SMILES: COc1cccc(C(=O)CC2OC(=O)c3ccccc3S2)c1]

Following the above-mentioned procedure, ketone (50 mg, 0.312 mmol, 1 eq.), DCM (4 ml), K₂CO₃ (2.1 mg, 5 mol %) and thiosalicylic acid (48.13 mg, 0.312 mmol, 1 eq.) were used to create the compound in question. The desired compound was isolated as a light yellow solid in 83% (82 mg, 0.260 mmol) yield by passing through a short plug of silica during a subsequent purification step using (20% EtOAc:Hexane) of the crude material. R_{f}=0.5 (EtOAc:Hexane in 25:75). ¹H NMR (400 MHz, CDCl₃): δ 8.19 (dd, J=8.4, 1.3 Hz, 1H, CHₐᵣ), 7.55-7.48 (m, 3H, CHₐᵣ), 7.40 (dd, J=8.0, 8.0 Hz, 1H, CHₐᵣ), 7.36-7.32 (m, 2H, CHₐᵣ), 7.16 (dd, J=8.4, 7.2 Hz, 1H, CHₐᵣ), 6.27 (dd, J=6.4, 5.6 Hz, 1H, CHOS), 3.92 (dd, J=6.4, 5.6 Hz, 1H, CHH), 3.86 (s, 3H, OMe), 3.53 (dd, J=6.0, 5.6 Hz, 1H, CHH). ¹³C NMR (100 MHz, CDCl₃): δ 193.81 (CO), 163.76 (CO), 160.13 (CHₐᵣ), 138.04 (CHₐᵣ), 137.48 (CHₐᵣ), 133.86 (CHₐᵣ), 132.83 (CHₐᵣ), 130.0 (CHₐᵣ), 127.85 (CHₐᵣ), 127.05 (CHₐᵣ), 124.24 (CHₐᵣ), 121.0 (CHₐᵣ), 120.69 (CHₐᵣ), 112.52 (CHₐᵣ), 78.34 (CHOS), 55.65 (CHH), 43.05 (OCH₃). HRMS (ESI) Data for C₁₇H₁₄O₄S [M+NH₄]⁺ Calculated=332.0951. Found=332.0968. IR(Vmax) (Diamond ATR) cm⁻¹: 3063, 2993, 2910, 2834, 1718, 1677, 1577, 688. M. P.=140-142° C.

### Example 2

### (2b) Synthesis of 2-(2-(3,4-dimethoxyphenyl)-2-oxoethyl)-4H-benzo[d][1,3]oxathiin-4-one

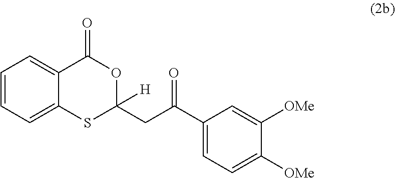 [SMILES: COc1ccc(C(=O)CC2OC(=O)c3ccccc3S2)cc1OC]

Following the above-mentioned procedure, ketone (50 mg, 0.209 mmol, 1 eq.), DCM (4 ml), K₂CO₃ (1.44 mg, 5 mol %) and thiosalicylic acid (32.24 mg, 0.209 mmol, 1 eq.) were used to create the compound in question. The desired compound was isolated as a yellow sticky solid in 76% (55 mg, 0.159 mmol) yield by passing through a short plug of silica during a subsequent purification step using (20% EtOAc:Hexane). R_{f}=0.5 (EtOAc:Hexane in 25:75). ¹H NMR (400 MHz, CDCl₃): δ 8.19 (dd, J=8.4 Hz, 1H), 7.59 (d, J=8.4 Hz, 1H), 7.54-7.48 (m, 2H), 7.36-7.32 (m, 2H), 6.92 (d, J=8.4 Hz, 1H), 6.25 (dd, J=6.4, 6.0 Hz, 1H), 3.97 (s, 3H), 3.94 (s, 3H), 3.88 (dd, J=16.8, 6.4 Hz, 1H), 3.51 (dd, J=16.8, 6.0 Hz, 1H). ¹³C NMR (100 MHz, CDCl₃): δ 192.44, 163.73, 154.19, 149.40, 138.11, 133.77, 132.75, 129.46, 127.80, 126.95, 124.23, 123.28, 110.36, 110.28, 78.64, 56.25, 56.13, 42.41. HRMS (ESI) Data for C₁₈H₁₆O₅S [M+H]⁺ Calculated=345.0791. Found=345.0797. IR(Vmax) (Diamond ATR) cm⁻¹: 2975, 2906, 1736, 1680, 1153, 1175, 1120, 1029.

### Example 3

### (2c) Synthesis of 2-(2-oxo-2-(p-tolyl)ethyl)-4H-benzo[d][1,3]oxathiin-4-one

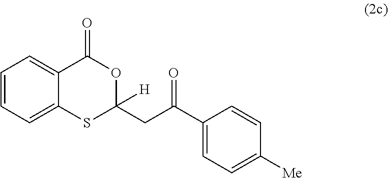 [SMILES: Cc1ccc(C(=O)CC2OC(=O)c3ccccc3S2)cc1]

From the above-mentioned procedures, ketone (50 mg, 0.346 mmol, 1 eq.), DCM (4 ml), K₂CO₃ (1.15 mg, 5 mol %) and thiosalicylic (53.43 mg, 0.346 mmol, 1 eq.) were used to create the compound in question. The desired compound was isolated as a yellow crystal in 73% (76 mg, 0.254 mmol) yield after the crude material was further purified by passing through a short plug of silica using (20% EtOAc:Hexane). R_{f}=0.5 (EtOAc:Hexane in 25:75). ¹H NMR (400 MHz, CDCl₃): δ 8.18 (d, J=8.4 Hz, 1H), 7.86 (d, J=8.3 Hz, 2H), 7.51-7.47 (m, 1H), 7.35-7.28 (m, 4H), 6.26 (dd, J=6.4, 6.0 Hz, 1H), 3.89 (dd, J=17.6, 6.4 Hz, 1H), 3.51 (dd, J=17.6, 6.0 Hz, 1H), 2.43 (s, 3H). ¹³C NMR (100 MHz, CDCl₃): δ 193.49, 163.78, 145.09, 138.08, 133.79, 133.79, 133.67, 132.75, 129.65, 128.79, 128.49, 127.81, 126.97, 124.21, 78.42, 42.74, 21.84. HRMS (ESI) Data for C17H14O3S [M+Na]⁺ Calculated=321.0556. Found=321.0558. IR(Vmax) (Diamond ATR) cm⁻¹: 3063, 2978, 2919, 1721, 1674, 1606, 1182, 1115, 1026. M.P.=152-153° C.

### Example 4

### (2d) Synthesis of 4-(2-(4-oxo-4H-benzo[d][1,3]oxathiin-2-yl)acetyl)benzonitrile

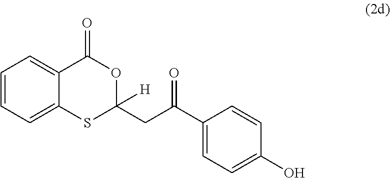 [SMILES: O=C(CC1OC(=O)c2ccccc2S1)c1ccc(O)cc1]

From the above-mentioned procedures, ketone (50 mg, 0.322 mmol, 1 eq.), DCM (4 ml), K₂CO₃ (2.2 mg, 5 mol %) and thiosalicylic acid (49.6 mg, 0.322 mmol, 1 eq.) were used to create the compound in question. The desired compound was isolated as a yellow solid in 73% (70 mg, 0.226 mmol) yield after the crude material underwent additional purification using a short plug of silica and a (20% EtOAc:Hexane) solvent mixture. R_{f}=0.5 (EtOAc:Hexane in 30:70). ¹H NMR (400 MHz, CDCl₃): δ 8.19 (dd, J=8.4, 8.0 Hz, 1H), 8.06 (d, J=8.4 Hz, 2H), 7.82 (d, J=8.8 Hz, 2H), 7.52 (dd, J=7.6, 7.6 Hz, 1H), 7.36 (dd, J=8.8, 6.8 Hz, 2H), 6.27 (dd, J=6.9, 5.2 Hz, 1H), 3.93 (dd, J=17.6, 6.9 Hz, 1H), 3.52 (dd, J=17.6, 5.2 Hz, 1H). ¹³C NMR (100 MHz, CDCl₃): δ 192.85, 163.47, 138.97, 137.68, 133.99, 132.90, 132.90, 132.86, 128.81, 128.81, 127.86, 127.22, 124.08, 117.78, 117.35, 77.84, 43.12. HRMS (ESI) Data for C₁₇H₁₁NO₃S [M+H]⁺ Calculated=310.0532. Found=310.0525. IR(Vmax) (Diamond ATR) cm⁻¹: 3228, 3058, 3040, 2937, 2872, 1627, 1174, 1118. M.P.=141-143° C.

### Example 5

### (2e) Synthesis of 2-(2-(4-methoxyphenyl)-2-oxoethyl)-4H-benzo[d][1,3]oxathiin-4-one

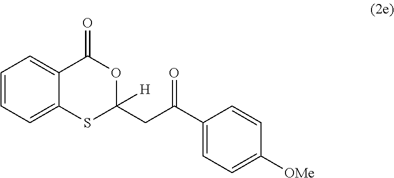 [SMILES: COc1ccc(C(=O)CC2OC(=O)c3ccccc3S2)cc1]

From the above-mentioned procedures, ketone (50 mg, 0.312 mmol, 1 eq.), DCM (4 ml), and K₂CO₃ (1.09 mg, 5 mol %) and thiosalicylic acid (48.13 mg, 0.312 mmol, 1 eq.) were used to create the compound in question. The desired compound was isolated as a yellow sticky liquid in 80% (79 mg, 0.251 mmol) yield after the crude material was further purified by passing through a short plug of silica using (20% EtOAc:Hexane). R_{f}=0.5 (EtOAc:Hexane in 25:75). ¹H NMR (400 MHz, CDCl₃): δ 8.17 (d, J=8.0 Hz, 1H), 7.95 (d, J=8.8 Hz, 2H), 7.49 (dd, J=8.0, 7.2 Hz, 1H), 7.33 (dd, J=7.6, 6.8 Hz, 2H), 6.96 (d, J=8.8 Hz, 2H), 6.26 (dd, J=6.0, 5.6 Hz, 1H), 3.88-3.83 (m, 4H), 3.49 (dd, J=17.2, 5.6 Hz, 1H). ¹³C NMR (100 MHz, CDCl₃): δ 192.36, 164.31, 163.85, 138.16, 133.80, 132.78, 130.78, 130.78, 129.27, 127.84, 126.99, 124.26, 114.16, 114.16, 78.60, 55.70, 42.52. HRMS (ESI) Data for C₁₇H₁₄O₄S [M+Na]⁺ Calculated=337.0505. Found=337.0506. IR(Vmax) (Diamond ATR) cm⁻¹: 2972, 2904, 2843, 1721, 1764, 1241, 1171, 1100.

### Example 6

### (2f) Synthesis of 2-(2-oxo-2-(3,4,5-trimethoxyphenyl)ethyl)-4H-benzol[d][1,3]oxathiin-4-one

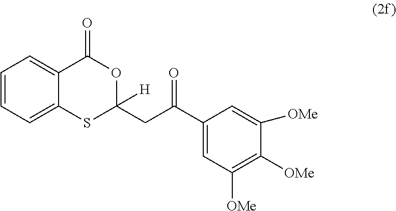 [SMILES: COc1cc(C(=O)CC2OC(=O)c3ccccc3S2)cc(OC)c1OC]

From the above-mentioned procedures, ketone (50 mg, 0.227 mmol, 1 eq.), DCM (4 ml), K₂CO₃ (2.13 mg, 5 mol %) and thiosalicylic acid (34.9 mg, 0.227 mmol, 1 eq.) were used to create the compound in question. The desired compound was isolated as white needle crystal in 68% (58 mg, 0.154 mmol) yield after the crude material underwent recrystallization in (MeOH:DCM 4:1) purification. R_{f}=0.5 (EtOAc:Hexane in 30:70). ¹H NMR (400 MHz, CDCl₃): δ 8.19 (d, J=8.8 Hz, 1H), 7.50 (ddd, J=7.6, 7.6, 1.6 Hz, 1H), 7.34-7.35 (m, 2H), 7.21 (s, 2H), 6.25 (dd, J=6.4, 6.0 Hz, 2H), 3.95-3.92 (m, 9H), 3.88 (dd, J=17.2, 6.4 Hz, 1H), 3.51 (dd, J=17.2, 6.0 Hz, 1H). ¹³C NMR (100 MHz, CDCl₃): δ 192.80, 163.73, 153.35, 153.35, 143.53, 138.01, 133.89, 132.84, 131.34, 127.87, 127.08, 124.20, 105.94, 78.50, 61.16, 56.52, 56.52, 56.52, 42.73. HRMS (ESI) Data for C₁₉H₁₈O₆S [M+Na]⁺ Calculated=397.0716. Found=397.0725. IR(Vmax) (Diamond ATR) cm⁻¹: 2990, 2916, 1730, 1677, 1153, 1130, 1109, 1029. M.P.=134-136° C.

### Example 7

### (2g) Synthesis of 2-(2-oxo-2-phenylethyl)-4H-benzo[d][1,3]oxathiin-4-one

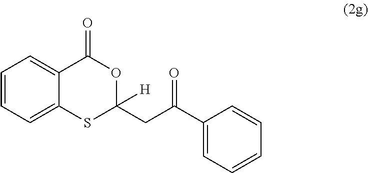 [SMILES: O=C(CC1OC(=O)c2ccccc2S1)c1ccccc1]

From the above-mentioned procedure, ketone (50 mg, 0.384 mmol, 1 eq.), DCM (4 ml), K₂CO₃ (2.65 mg, 5 mol %) and thiosalicylic acid (59.23 mg, 0.384 mmol, 1 eq.) were used to create the compound in question. The desired compound was isolated as a white solid in 82% (88 mg, 0.309 mmol) yield after the crude material was further purified by passing through a short plug of silica using (15% Hexane:EtOAc). R_{f}=0.5 (EtOAc:Hexane in 20:80). ¹H NMR (400 MHz, CDCl₃): δ 8.19 (dd, J=8.4, 2.0 Hz, 1H), 7.97 (d, J=6.8 Hz, 2H), 7.62 (dd, J=7.6, 7.2, 2.0 Hz, 1H), 7.53-7.48 (m, 3H), 7.36-7.32 (m, 2H), 6.28 (dd, J=6.4, 6.0 Hz, 1H), 3.93 (dd, J=17.2, 6.4 Hz, 1H), 3.54 (dd, J=17.2, 6.0 Hz, 1H). ¹³C NMR (100 MHz, CDCl₃): δ 193.96, 163.79, 138.05, 136.14, 134.14, 133.86, 132.84, 129.03, 129.03, 128.41, 128.41, 127.87, 127.06, 124.26, 78.35, 42.92. HRMS (ESI) Data for C₁₆H₁₂O₃S [M+NH₄]⁺ Calculated=302.0845. Found=302.0853. IR(Vmax) (Diamond ATR) cm⁻¹: 2966, 2919, 2852, 1730, 1680, 1105. M.P.=128-130° C.

### Example 8

### (2h) Synthesis of 2-(2-(4-chlorophenyl)-2-oxoethyl)-4H-benzo[d][1,3]oxathiin-4-one

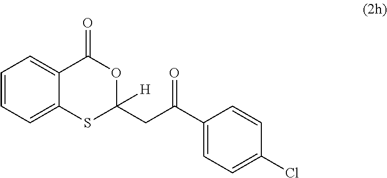 [SMILES: O=C(CC1OC(=O)c2ccccc2S1)c1ccc(Cl)cc1]

From the above-mentioned procedures, ketone (50 mg, 0.303 mmol, 1 eq.), DCM (4 ml), K₂CO₃ (2.0 mg, 5 mol %) and thiosalicylic acid (46.71 mg, 0.303 mmol, 1 eq.) were used to create the compound in question. The desired compound was isolated as a light yellow sticky liquid with 73% (70 mg. 0.222 mmol) yield after the crude material was further purified by passing through a short plug of silica using (20% EtOAc:Hexane). R_{f}=0.4 (EtOAc:Hexane in 20:80). ¹H NMR (400 MHz, CDCl₃): δ 8.19 (d, J=8.4, 1.2 Hz, 1H), 7.93-7.89 (m, 2H), 7.52-7.46 (m, 3H), 7.36-7.32 (m, 2H), 6.26 (dd, J=5.6, 5.2 Hz, 1H), 3.89 (dd, J=17.2, 5.6 Hz, 1H), 3.48 (dd, J=17.2, 5.2 Hz, 1H). ¹³C NMR (100 MHz, CDCl₃): δ 192.81, 163.66, 140.73, 137.92, 134.49, 133.91, 132.87, 129.81, 129.81, 129.39, 129.39, 127.86, 127.12, 124.20, 78.17, 42.86. HRMS (ESI) Data for C₁₆H₁₃ClO₂S [M+H]⁺ Calculated=319.0190. Found=319.0118. IR(Vmax) (Diamond ATR) cm⁻¹: 3058, 2975, 1721, 1677, 1132, 1106.

### Example 9

### (2i) Synthesis of 2-(2-(4-fluorophenyl)-2-oxoethyl)-4H-benzo[d][1,3]oxathiin-4-one

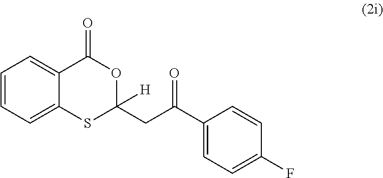 [SMILES: O=C(CC1OC(=O)c2ccccc2S1)c1ccc(F)cc1]

From the above-mentioned procedures, ketone (50 mg, 0.337 mmol, 1 eq.), DCM (4 ml), K₂CO₃ (2.32 mg, 5 mol %) and thiosalicylic acid (52.03 mg, 0.337 mmol, 1 eq.) were used to create the compound in question. The crude material was further purified by passing through short plug of silica using (20% EtOAc:Hexane) and the desired product was isolated as light yellow solid in 77% (79 mg, 0.261 mmol) yield. ¹H NMR (400 MHz, CDCl₃): δ 8.19 (d, J=8.4 Hz, 1H), 8.04-7.99 (m, 2H), 7.51 (ddd, J=7.6, 7.6, 1.2 Hz, 1H), 7.37-7.33 (m, 2H), 7.28 (d, J=8.8 Hz, 2H), 6.26 (dd, J=6.8, 5.2 Hz, 1H), 3.88 (dd, J=17.2, 6.4 Hz, 1H), 3.50 (dd, J=17.2, 5.6 Hz, 1H). ¹⁹F=−103.33. 13C NMR (100 MHz, CDCl₃): δ 192.38, 167.64, 165.10, 163.67, 137.94, 133.88, 132.84, 131.18, 131.09, 127.85, 127.08, 124.20, 116.32, 116.10, 78.24, 42.79. HRMS (ESI) Data for C₁₆H₁₁FO₃S [M+NH₄]⁺ Calculated=320.0751. Found= 320.0752. IR(Vmax) (Diamond ATR) cm⁻¹: 2904, 2849, 1721, 1677, 1230, 1156, 1103. M.P.=147-149° C.

### Example 10

### (2j) Synthesis of 2-(2-(4-bromophenyl)-2-oxoethyl)-4H-benzo[d][1,3]oxathiin-4-one

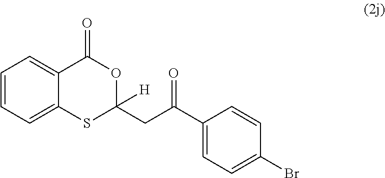 [SMILES: O=C(CC1OC(=O)c2ccccc2S1)c1ccc(Br)cc1]

The title compound was synthesized by following general procedure B, ketone (50 mg, 0.239 mmol, 1 eq.), DCM (4 ml), K₂CO₃ (1.65 mg, 5 mol %) and thiosalicylic acid (36.84 mg, 0.239 mmol, 1 eq.). The crude material was further purified by passing through short plug of silica using (20% EtOAc:Hexane), the desired compound was isolated as light brown sticky liquid in 86% (75 mg, 0.206 mmol) yield. R_{f}=0.4 (EtOAc:Hexane in 30:70). ¹H NMR (400 MHz, CDCl₃): δ 8.18 (d, J=8.2 Hz, 1H), 7.85-7.82 (m, 2H), 7.66-7.64 (m, 2H), 7.52-7.48 (m, 1H), 7.36-7.32 (m, 2H), 6.26 (dd, J=6.4, 5.6 Hz, 1H), 3.88 (dd, J=17.2, 6.4 Hz, 1H), 3.49 (dd, J=17.2, 5.6 Hz, 1H). ¹³C NMR (100 MHz, CDCl₃): δ 192.99, 163.63, 137.89, 134.86, 133.89, 132.86, 132.37, 132.22, 131.16, 129.86, 129.49, 127.85, 127.11, 124.18, 78.12, 42.82. HRMS (ESI) Data for C₁₆H₁₁BrO₃S [M+K]⁺ Calculated=400.9244, Found=400.9227. IR(Vmax) (Diamond ATR) cm⁻¹: 3052, 2957, 2916, 1677, 1642, 1177.

### Example 11

### (2k) Synthesis of 2-(2-(4-nitrophenyl)-2-oxoethyl)-4H-benzo[d][1,3]oxathiin-4-one

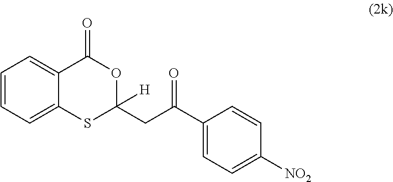 [SMILES: O=C(CC1OC(=O)c2ccccc2S1)c1ccc([N+](=O)[O-])cc1]

The title compound was synthesized by following general procedure B, ketone (50 mg, 0.287 mmol, 1 eq.), DCM (4 ml), K2CO3 (1.14 mg, 5 mol %) and thiosalicylic acid (44.26 mg, 0.287 mmol, 1 eq.). The crude material was further purified by passing through short plug of silica using (30% Hexan:EtOAc) and the desired product was isolated as yellow solid in 80% (76 mg, 0.230 mmol) yield. R_{f}=0.4 (EtOAc:Hexane in 40:60). ¹H NMR (400 MHz, CDCl₃): δ 8.36 (d, J=9.2 Hz, 2H), 8.19 (d, J=8.4 Hz, 1H), 8.14 (d, J=8.8 Hz, 2H), 7.52 (dd, J=8.0, 7.2 Hz, 1H), 7.39-7.32 (m, 2H), 6.28 (dd, J=6.8, 4.8 Hz, 1H), 3.97 (dd, J=17.2, 6.8 Hz, 1H), 3.55 (dd, J=17.2, 4.8 Hz, 1H). ¹³C NMR (100 MHz, CDCl₃): δ 192.68, 163.42, 151.00, 140.50, 137.68, 134.02, 132.98, 129.52, 129.52, 127.90, 127.28, 127.28, 124.27, 124.16, 77.84, 43.40. HRMS (ESI) Data for C₁₆H₁₁NO₅S [M+K]⁺ Calculated=367.999. Found=367.9818. IR(Vmax) (Diamond ATR) cm⁻¹: 3108, 3078, 3052, 1718, 1692, 1238, 1215, 1109, 1009. M.P.=143-146° C.

### Example 12

### (21) Synthesis of 2-(2-(4-(allyloxy)phenyl)-2-oxoethyl)-4H-benzo[d][1,3]oxathiin-4-one

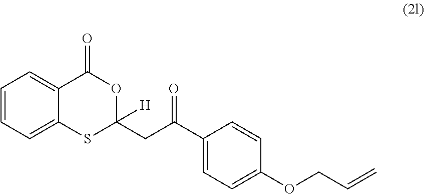 [SMILES: C=CCOc1ccc(C(=O)CC2OC(=O)c3ccccc3S2)cc1]

The title compound was synthesized by following general procedure B, ketone (50 mg, 0.268 mmol, 1 eq.), DCM (4 ml), K2CO3 (1.85 mg, 5 mol %) and thiosalicylic acid (41.32 mg, 0.268 mmol, 1 eq.). The crude material was further purified by passing through short plug of silica using (20% EtOAc:Hexane) and the desired compound was isolated as white solid in 80% (73 mg, 0.214 mmol) yield. R_{f}=0.5 (EtOAc:Hexane in 25:75). ¹H NMR (400 MHz, CDCl3): δ 8.18 (dd, J=8.4, 2.0 Hz, 1H), 7.94 (d, J=8.8 Hz, 2H), 7.49 (ddd, J=7.6, 7.6, 2.0 Hz, 1H), 7.36-7.33 (m, 2H), 6.97 (d, J=8.8 Hz, 2H), 6.25 (dd, J=6.4, 6.0 Hz, 1H), 6.09-6.00 (m, 1H), 5.42 (ddt, J=17.2, 2.4, 1.6 Hz, 1H), 5.33 (ddt, J=10.2, 2.4, 1.6 Hz, 1H), 4.62 (dt, J=5.2, 1.2 Hz, 2H), 3.86 (dd, J=16.4, 6.4 Hz, 1H), 3.49 (dd, J=16.4, 6.0 Hz, 1H). ¹³C NMR (100 MHz, CDCl₃): δ 192.36, 163.85, 163.31, 138.16, 133.81, 132.79, 132.43, 130.76, 130.76, 129.36, 127.85, 126.99, 124.27, 118.51, 114.86, 114.86, 78.59, 69.11, 42.53. HRMS (ESI) Data for C₁₉H₁₆O₄S [M+Na]⁺ Calculated=379.0401. Found=379.0415. IR(Vmax) (Diamond ATR) cm⁻¹: 2921, 2849, 1728, 1665, 1179, 1107

### Example 13

### (2m) Synthesis of 2-(2-oxo-2-(3-phenoxyphenyl)ethyl)-4H-benzo[d][1,3]oxathiin-4-one

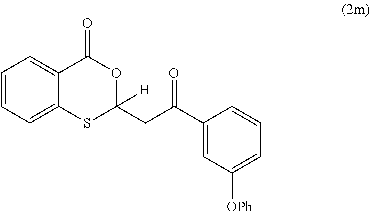 [SMILES: O=C(CC1OC(=O)c2ccccc2S1)c1cccc(Oc2ccccc2)c1]

The title compound was synthesized by following general procedure B, ketone (50 mg, 0.224 mmol, 1 eq.), DCM (4 ml), K2CO3 (1.54 mg, 5 mol %) and thiosalicylic acid (34.68 mg, 0.224 mmol, 1 eq.). The crude material was further purified by passing through short plug of silica using (20% EtOAc:Hexane) and the desired compound was isolated as yellow sticky liquid in 72% (60 mg, 0.162 mmol) yield. R_{f}=0.4 (EtOAc:Hexane in 25:75). ¹H NMR (400 MHz, CDCl3): δ 8.18 (dd, J=8.4 Hz, 1H), 7.68 (d, J=8.0, 7.6, 7.6 Hz, 1H), 7.57 (dd, J=2.0, 2.0 Hz, 1H), 7.52-7.43 (m, 2H), 7.39-7.32 (m, 4H), 7.27-7.26 (m, 1H), 7.16 (dd, J=7.6, 7.2 Hz, 1H), 7.04-7.00 (m, 2H), 6.25 (dd, J=6.4, 6.0 Hz, 1H), 3.88 (dd, J=17.2, 6.4 Hz, 1H), 3.48 (dd, J=17.2, 6.0 Hz, 1H). ¹³C NMR (100 MHz, CDCl₃): δ 193.34, 163.70, 158.21, 156.43, 137.98, 137.81, 133.86, 132.82, 130.39, 130.39, 130.18, 130.18, 127.85, 127.85, 127.06, 124.23, 124.19, 122.99, 119.43, 117.92, 78.22, 43.01. HRMS (ESI) Data for C₂₂H₁₆O₄S [M+H]⁺ Calculated=377.0842. Found=377.0852. IR(Vmax) (Diamond ATR) cm⁻¹: 2978, 2922, 1686, 1596, 1525, 1216, 1171, 1133, 1114, 1039.

### Example 14

### (2n) Synthesis of 2-(2-(6-bromobenzo[d][1,3]dioxol-5-yl)-2-oxoethyl) 4Hbenzo[d][1,3]oxathiin-4-one

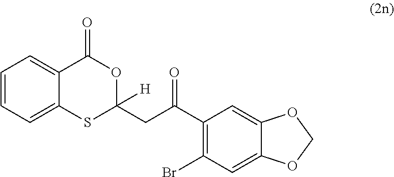 [SMILES: O=C(CC1OC(=O)c2ccccc2S1)c1cc2c(cc1Br)OCO2]

The title compound was synthesized by following general procedure B, ketone (50 mg, 0.197 mmol, 1 eq.), DCM (4 ml), K2CO3 (1.36 mg, 5 mol %) and thiosalicylic acid (30.46 mg, 0.197 mmol, 1 eq.). The crude material was further purified through recrystallization in MeOH:DCM (4:1) and the desired compound was isolated as yellow solid in 79% (64 mg, 0.157 mmol) yield. R_{f}=0.5 (EtOAc:Hexane in 30:70). ¹H NMR (400 MHz, CDCl₃): δ 8.17 (dd, J=8.4 Hz, 1H), 7.49 (ddd, J=8.0, 7.2, 1.2 Hz, 1H), 7.35 (m, 2H), 7.06 (s, 2H), 6.18 (dd, J=6.8, 5.6 Hz, 1H), 6.06 (s, 2H), 3.85 (dd, J=17.2, 6.8 Hz, 1H), 3.57 (dd, J=17.2, 5.6 Hz, 1H). ¹³C NMR (100 MHz, CDCl₃): δ 195.83, 163.64, 151.20, 147.83, 138.00, 133.87, 133.22, 132.85, 127.86, 127.08, 124.24, 114.13, 112.45, 109.74, 102.80, 78.56, 46.57. HRMS (ESI) Data for C₁₇H₁₁BrO₅S [M+Na]⁺ Calculated=428.9403. Found=428.9496. IR(Vmax) (Diamond ATR) cm⁻¹: 2934, 2916, 1715, 1698, 1612, 1592, 1252, 1121, 1014 cm⁻¹. M.P.=141-143° C.

### Example 15

### (2o) Synthesis of 2-(2-(2-bromo-5-methoxyphenyl)-2-oxoethyl)-4H-benzo[d][1,3]oxathiin-4-one

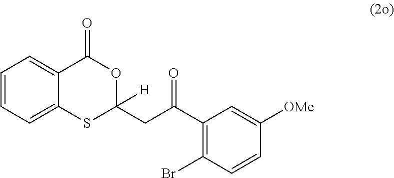 [SMILES: COc1ccc(Br)c(C(=O)CC2OC(=O)c3ccccc3S2)c1]

The title compound was synthesized by following general procedure B, ketone (50 mg, 0.209 mmol, 1 eq.), DCM (4 ml), K2CO3 (1.44 mg, 5 mol %) and thiosalicylic acid (32.24 mg, 0.209 mmol, 1 eq.). The crude material was further purified by passing through short plug of silica using (20% EtOAc:Hexane) and the desired compound was isolated as yellow sticky liquid in 82% (67.5 mg, 0.171 mmol) yield. R_{f}=0.5 (EtOAc:Hexane:Et3N in 25:74:1). ¹H NMR (400 MHz, CDCl₃): δ 8.18 (d, J=7.6 Hz, 1H), 7.52-7.48 (m, 2H), 7.36-7.31 (m, 2H), 7.02 (s, 1H), 6.89 (dd, J=8.8, 8.4 Hz, 1H), 6.19 (dd, J=6.8, 6.0 Hz, 1H), 3.83 (dd, J=17.2, 6.4 Hz, 1H), 3.82 (m, 3H), 3.61 (dd, J=17.2, 6.0 Hz, 1H). ¹³C NMR (100 MHz, CDCl₃): δ 197.62, 163.58, 159.18, 141.06, 137.95, 134.80, 133.89, 132.85, 127.85, 127.09, 124.20, 119.02, 114.42, 109.15, 78.20, 55.80, 46.74. HRMS (ESI) Data for C₁₇H₁₃BrO₄S [M+H]⁺ Calculated=392.9791, 394.9771. Found=392.9809, 394.9793. IR(Vmax) (Diamond ATR) cm⁻¹: 3069, 2931, 2910, 1721, 1698, 1238, 1106, 1006.

### Example 16

### (2p) Synthesis of 4-(2-(4-oxo-4H-benzo[d][1,3]oxathiin-2-yl)acetyl)phenyl 4-methylbenzenesulfonate

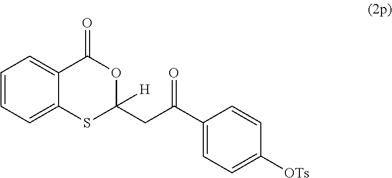 [SMILES: Cc1ccc(S(=O)(=O)Oc2ccc(C(=O)CC3OC(=O)c4ccccc4S3)cc2)cc1]

The title compound was synthesized by following general procedure B, ketone (50 mg, 0.166 mmol, 1 eq.), DCM (4 ml), K2CO3 (1.15 mg, 5 mol %) and thiosalicylic acid (25.6 mg, 0.166 mmol, 1 eq.). The crude material was further purified by passing through short plug of silica using (20% EtOAc:Hexane) and the desired product was isolated as yellow liquid in 72% (55 mg, 0.121 mmol) yield. R_{f}=0.5 (EtOAc:Hexane in 25:75). ¹H NMR (400 MHz, CDCl₃): δ 8.17 (d, J=8.0 Hz, 1H), 7.91 (d, J=8.8 Hz, 2H), 7.71 (d, J=8.4 Hz, 2H), 7.52-7.45 (m, 2H), 7.36-7.32 (m, 3H), 7.12 (d, J= 8.8 Hz, 2H), 6.26-6.23 (dd, J=5.6, 6.4 Hz, 1H), 3.88 (dd, J=6.8, 6.8 Hz, 1H), 3.44 (dd, J=5.6, 5.2 Hz, 1H), 2.46 (s, 3H). ¹³C NMR (100 MHz, CDCl₃): δ 192.61, 163.65, 153.74, 146.05, 146.05, 137.87, 134.68, 133.92, 132.87, 132.10, 130.20, 130.13, 130.08, 128.64, 128.49, 127.86, 127.14, 124.17, 122.97, 122.23, 78.09, 42.87, 21.90. HRMS (ESI) Data for C₂₃H₁₈O₆S₂ [M+H]⁺ Calculated=455.0618. Found=455.0616. IR(Vmax) (Diamond ATR) cm⁻¹: 2957, 2925, 2854, 1730, 1652, 1636, 1147, 1174, 1056.

### Example 17

### (2q) Synthesis of 2-(2-cyclopropyl-2-oxoethyl)-4H-benzo[d][1,3]oxathiin-4-one

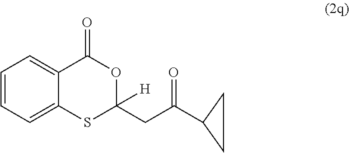 [SMILES: O=C1OC(CC(=O)C2CC2)Sc2ccccc21]

The title compound was synthesized by following general procedure B, ketone (50 mg, 0.531 mmol, 1 eq.), DCM (4 ml), K2CO3 (3.6 mg, 5 mol %) and thiosalicylic acid (81.91 mg, 0.531 mmol, 1 eq.). The crude material was further purified by passing through short plug of silica using (20% EtOAc:Hexane) and the desired compound was isolated as sticky liquid in 76% (101 mg, 0.406 mmol) yield. R_{f}=0.5 (EtOAc:Hexane in 20:80). ¹H NMR (400 MHz, CDCl₃): δ 8.15 (d, J=8.4 Hz, 1H), 7.48 (dd, J=7.6, 7.6 Hz, 1H), 7.34-7.30 (m, 2H), 6.07 (dd, J=6.4, 5.6 Hz, 1H), 3.46 (dd, J=17.2, 6.4 Hz, 1H), 3.15 (dd, J=17.2, 5.6 Hz, 1H), 1.98 (m, 1H), 1.16-1.11 (m, 2H), 1.00-0.97 (m, 2H). ¹³C NMR (100 MHz, CDCl₃): δ 204.56, 163.80, 138.06, 133.80, 132.78, 127.79, 126.99, 124.21, 77.93, 47.12, 29.82, 21.49, 11.85. HRMS (ESI) Data for C₁₃H₁₂O₃S [M+K]⁺ Calculated=287.0139. Found=287.0156. IR(Vmax) (Diamond ATR) cm⁻¹: 3013, 2957, 2928, 1724, 1692, 1194, 1109.

### Example 18

### (2r) Synthesis of 2-(2-oxo-4-phenylbutyl)-4H-benzo[d][1,3]oxathiin-4-one

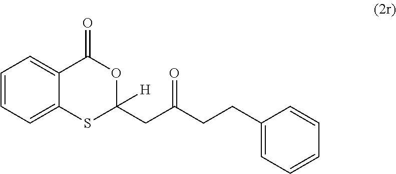 [SMILES: O=C(CCc1ccccc1)CC1OC(=O)c2ccccc2S1]

The title compound was synthesized by following general procedure B, ketone (50 mg, 0.367 mmol, 1 eq.), DCM (4 ml), K2CO3 (2.58 mg, 5 mol %) and thiosalicylic acid (56.65 mg, 0.367 mmol, 1 eq.). The crude material was further purified by passing through short plug of silica using (20% Hexan/EtOAc) and the desired compound was isolated as white solid in 80% (92 mg, 0.294 mmol) yield. R_{f}=0.5 (EtOAc:Hexane in 15:85). ¹H NMR (400 MHz, CDCl₃): δ 8.14 (d, J=7.6 Hz, 1H), 7.47 (dd, J=7.6, 7.2 Hz, 1H), 7.33-7.26 (m, 4H), 7.19 (dd, J=7.6, 7.2 Hz, 3H), 6.04 (dd, J=7.2, 4.8 Hz, 1H), 3.26 (dd, J=17.2, 7.2 Hz, 1H), 2.96-2.90 (m, 3H), 2.86-2.81 (m, 2H). ¹³C NMR (100 MHz, CDCl₃): δ 203.69, 163.60, 140.43, 137.88, 133.79, 132.73, 128.67, 128.35, 128.35, 127.73, 127.73, 126.99, 126.37, 124.07, 77.79, 46.60, 45.20, 29.41. HRMS (ESI) Data for C₁₈H₁₆O₃S [M+NH4]⁺ Calculated=330.1158. Found=330.1157. IR(Vmax) (Diamond ATR) cm⁻¹: 3028, 2955, 2928, 2902, 1718, 1730, 1271, 1106, 1032. M. P.=48-50° C.

### Example 19

### (2s) Synthesis of 2-(2-(cyclohex-2-en-1-yl)-2-oxoethyl)-4H-benzo[d][1,3]oxathiin-4-one

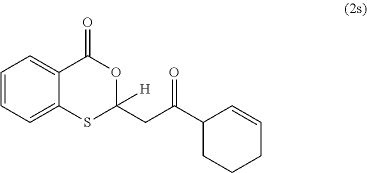 [SMILES: O=C1OC(CC(=O)C2C=CCCC2)Sc2ccccc21]

The title compound was synthesized by following general procedure B, ketone (50 mg, 0.372 mmol, 1 eq.), DCM (4 ml), K₂CO₃ (2.57 mg, 5 mol %) and thiosalicylic acid (57.35 mg, 0.372 mmol, 1 eq.). The crude material was further purified through recrystallization, and was isolated as white crystal in 80% (92 mg, 0.294 mmol) yield. R_{f}=0.8 (EtOAc:Hexane in 5:95). ¹H NMR (400 MHz, CDCl₃): δ 8.16 (d, J=8.4 Hz, 1H), 7.49 (dd, J=7.6, 7.6 Hz, 1H), 7.35-7.31 (m, 2H), 6.08 (dd, J=7.2, 5.2 Hz, 1H), 5.70 (s, 2H), 3.48-3.35 (m, 1H), 3.10-2.99 (m, 1H), 2.71-2.64 (m, 1H), 2.23-1.98 (m, 5H), 1.67-1.59 (m, 1H). ¹³C NMR (100 MHz, CDCl₃): δ 207.12, 163.70, 137.99, 133.80, 132.78, 127.76, 127.00, 126.73, 124.88, 124.20, 78.10, 47.31, 44.59, 26.50, 24.57, 24.17. HRMS (ESI) Data for C₁₆H₁₆O₃S [M+Na]⁺ Calculated=311.0712. Found=311.0723. IR(Vmax) (Diamond ATR) cm⁻¹: 2920, 1717, 1676, 1572, 1213, 1102, 1060, 1030. M. P.=138-140° C.

### Example 20

### (2t) Synthesis of 2-(2-(2-bromophenyl)-2-oxoethyl)-4H-benzo[d][1, 3]oxathiin-4-one

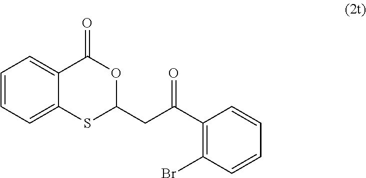 [SMILES: O=C(CC1OC(=O)c2ccccc2S1)c1ccccc1Br]

The title compound was synthesized by following general procedure B, ketone (50 mg, 0.237 mmol, 1 eq.), DCM (4 ml), K₂CO₃ (1.6 mg, 5 mol %) and thiosalicylic acid (36.87 mg, 0.237 mmol, 1 eq.). The crude material was further purified by passing through short plug of silica using (20% EtOAc:Hexane) and the title compound was isolated as yellow sticky oil in 78% (67 mg, 0.184 mmol) yield. R_{f}=0.5 (EtOAc:Hexane in 25:75). ¹H NMR (400 MHz, CDCl₃): δ 8.17 (d, J=8.4, 1.6 Hz, 1H), 7.63 (dd, J=8.0, 1.2 Hz, 1H), 7.52-7.47 (m, 2H), 7.41 (ddd, J=7.2, 7.2, 1.2 Hz, 1H), 7.36-7.31 (m, 3H), 6.20 (dd, J=6.8, 5.6 Hz, 1H), 3.85 (dd, J=17.2, 5.6 Hz, 1H), 3.60 (dd, J=17.2, 6.8 Hz, 1H). ¹³C NMR (100 MHz, CDCl₃): δ 197.59, 163.57, 140.29, 137.91, 134.05, 133.87, 132.84, 132.64, 129.41, 127.87, 127.84, 127.08, 124.20, 119.10, 78.20, 46.69. HRMS (ESI) Data for C₁₆H₁₁BrO₃S [M+H]⁺ Calculated=362.9685. Found=362.9693. IR(Vmax) (Diamond ATR) cm⁻¹: 3052, 2934, 2831, 1680, 1633, 1262, 1200, 1174.

### Example 21

### (1) Synthesis of 2-(5-(tert-butyl)benzo[d][1,3]dioxol-2-yl)-1-(3-methoxyphenyl)ethan-1-one

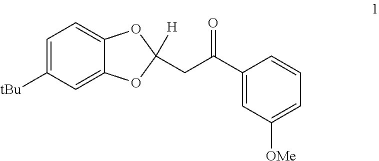 [SMILES: COc1cccc(C(=O)CC2Oc3ccc(C(C)(C)C)cc3O2)c1]

Title compound was synthesized using the general procedure B, starting from ynone (50 mg, 0.312 mmol, 1 eq.), K₂CO₃ (43.14 mg, 0.312 mmol, 1 eq.), DCM (4 ml) and catechol derivative (51.8 mg, 0.312 mmol, 1 eq.). The desired compound was purified through column chromatography (30% EtOAc:Hexane) and isolated as light brown sticky liquid in 91.0% (93.2 mg, 0.285 mmol) yield. R_{f}=0.4 (EtOAc:Hexane in 30:70). ¹H NMR (400 MHz, CDCl₃): δ 7.52 (m, 2H), 7.38 (dd, J=8.0, 8.0 Hz, 1H), 7.14 (ddd, J=8.4, 8.0, 2.8 Hz, 1H), 6.89 (d, J=2.0 Hz, 1H), 6.83 (dd, J=8.0, 2.0 Hz, 1H), 6.71 (t, J=4.8 Hz, 1H), 3.86 (s, 3H), 3.61 (d, J=4.8 Hz, 2H), 1.28 (s, 9H). ¹³C NMR (100 MHz, CDCl₃): δ 195.40, 160.04, 147.17, 145.54, 144.89, 137.98, 129.84, 121.08, 120.41, 117.97, 112.43, 108.50, 107.86, 106.59, 55.57, 43.99, 34.73, 31.70, 31.70, 31.70. HRMS (ESI) DATA for C₂₀H₂₂O₄[M+H]⁺ Calculated=327.1591. Found (Dimer)=655.2002. IR(Vmax) (Diamond ATR) cm⁻¹: 2957, 2907, 1680, 1595, 1586, 1489, 1412.

### Example 22

### (2) Synthesis of 2-(4,6-di-tert-butyl benzo[d][1,3]dioxol-2-yl)-1-(3-methoxyphenyl)ethan-1-one

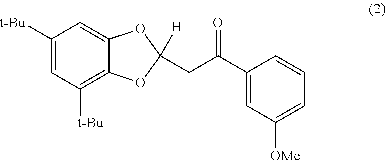 [SMILES: COc1cccc(C(=O)CC2Oc3cc(C(C)(C)C)cc(C(C)(C)C)c3O2)c1]

Title compound was synthesized using the general procedure B, starting from ynone (50 mg, 0.312 mmol, 1 eq.), K₂CO₃ (43.14 mg, 0.312 mmol, 1 eq.), DCM (4 ml) and 4,6-di-tert-butyl catechol (69.4 mg, 0.312 mmol, 1 eq.), and the desired compound was purified through column chromatography (20% EtOAc:Hexane) and isolated as pale yellow liquid in 92% (110.2 mg, 0.287 mmol) yield. R_{f}=0.6 (EtOAc:Hexane in 20:80). ¹H NMR (400 MHz, CDCl₃): 7.58-7.53 (m, 2H), 7.41 (dd, J=8.0, 8.0 Hz, 1H), 7.16 (ddd, J=8.4, 2.8, 1.2 Hz, 1H), 6.77 (dd, J=11.2, 2.0 Hz, 2H), 6.68 (t, J=4.8 Hz, 1H), 3.86 (s, 3H), 3.6 (d, J=4.8 Hz, 2H), 1.32 (s, 9H), 1.27 (s, 9H). ¹³C NMR (100 MHz, CDCl₃): δ 195.59, 160.05, 147.07, 144.80, 142.25, 138.16, 132.00, 129.83, 121.23, 120.38, 115.54, 112.45, 107.80, 104.48, 55.62, 44.09, 34.93, 34.21, 31.81, 31.81, 31.81, 29.63, 29.63, 29.63. HRMS (ESI) Data for C₂₄H₃₀O₄[M+Na⁺] Calculated=405.2054. Found=405.2036. IR(Vmax) (Diamond ATR) cm⁻¹: 1380, 1736, 1598, 1580, 1483, 1253, 3066, 2952.

### Example 23

### (3) Synthesis of 2-(4-methoxybenzo[d][1,3]dioxol-2-yl)-1-(3-methoxyphenyl)ethan-1-one

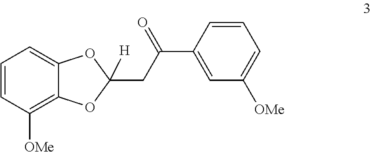 [SMILES: COc1cccc(C(=O)CC2Oc3cccc(OC)c3O2)c1]

Title compound was synthesized using the general procedure B, starting from ynone (50 mg, 0.312 mmol, 1 eq.), K₂CO₃ (43.14 mg, 0.312 mmol, 1 eq.), DCM (4 ml) and 3-methoxy catechol (43.7 mg, 0.312 mmol, 1 eq.). The desired compound was purified through column chromatography (20% EtOAc:Hexane) and isolated as sticky liquid in 89% (83 mg, 0.279 mmol) yield. R_{f}=0.5 (EtOAc:Hexane in 20:80). ¹H NMR (400 MHz, CDCl₃): 7.51 (tt, J=7.6, 1.6 Hz, 2H), 7.37 (t, J=7.6 Hz, 1H), 7.13 (dd, J=8.4, 2.8 Hz, 1H), 6.76 (dt, J=9.6, 8.4 Hz, 2H), 6.51 (td, J=7.2, 1.2 Hz, 2H), 3.88 (s, 3H), 3.85 (s, 3H), 3.64 (dd, J=5.2, 2.8 Hz, 2H). ¹³C NMR (100 MHz, CDCl₃): δ 195.09, 160.03, 148.53, 144.08, 137.94, 135.05, 129.84, 122.13, 121.04, 120.44, 112.37, 109.07, 107.48, 102.61, 56.57, 56.50, 43.86. HRMS (ESI) DATA for C₁₇H₁₆O₅ [M+H]⁺ Calculated=301.1071. Found=301.1083. IR (Vmax) (Diamond ATR) cm⁻¹: 2830, 1640, 1433, 1683, 1506, 1468, 1302, 2916, 2854

### Example 24

(4) Synthesis of 2-(2-(3-methoxyphenyl)-2-oxoethyl) benzo[d][1,3]dioxole-5-carbonitrile:

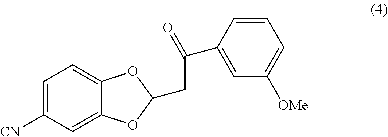

Title compound was synthesized using the general procedure B starting from ynone (50 mg, 0.312 mmol, 1 eq.), K₂CO₃ (43.14 mg, 0.312 mmol, 1 eq.), acetone (4 ml) and catechol derivative (42.18 mg, 0.312 mmol, 1 eq.). The desired compound was purified through column chromatography (15% EtOAc:Hexane) and isolated as white solid in 82% (76.2 mg, 0.258 mmol) yield. R_{f}=0.5 (EtOAc:Hexane in 25:75). ¹H NMR (400 MHz, CDCl3): δ 7.51-7.47 (m, 2H), 7.39 (ddd, J=15.6, 8.0, 2.8 Hz, 1H), 7.21-7.13 (m, 2H), 7.01 (m, 1H), 6.86-6.83 (m, 2H), 3.86 (s, 3H), 3.65 (d, J=4.8 Hz, 2H). ¹³C NMR (100 MHz, CDCl3): δ 194.53, 160.13, 151.31, 147.83, 137.57, 129.97, 128.35, 120.95, 120.59, 118.98, 112.50, 111.63, 110.46, 109.37, 105.13, 55.62, 43.71. HRMS (ESI) DATA for C₁₇H₁₃NO₄ [M+H]⁺ Calculated=296.0917. Found=296.0939. IR(Vmax) (Diamond ATR) cm⁻¹: 3058, 2949, 2837, 2219, 1683, 1577, 1433. M.P.=100-101° C.

### Example 25

### (5) Synthesis of 1-(3-methoxyphenyl)-2-(5-methylbenzo[d][1,3]dioxol-2-yl)ethan-1-one

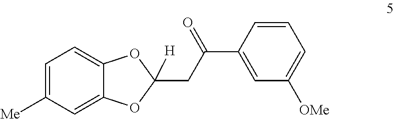 [SMILES: COc1cccc(C(=O)CC2Oc3ccc(C)cc3O2)c1]

Title compound was synthesized using the general procedure B, starting from ynone (50 mg, 0.312 mmol, 1 eq.), K₂CO₃ (43.14 mg, 0.312 mmol, 1 eq.), DCM (4 ml) and catechol derivative (38.75 mg, 0.312 mmol, 1 eq.). The desired compound was purified through column chromatography (20% EtOAc:Hexane) and isolated as yellow sticky liquid in 78% (69.2 mg, 0.243 mmol) yield. R_{f}=0.5 (EtOAc:Hexane in 20:80). ¹H NMR (400 MHz, DCl3): δ 7.53-7.49 (m, 2H), 7.38 (t, J=7.6 Hz, 1H), 7.14 (dd, J=8.4, 3.6 Hz, 1H), 6.71-6.68 (m, 2H), 6.64-6.60 (m, 2H), 3.86 (s, 3H), 3.60 (d, J=4.8 Hz, 2H), 2.27 (s, 3H). ¹³C NMR (100 MHz, CDCl₃): δ 195.43, 160.08, 147.27, 145.09, 138.00, 131.68, 129.87, 121.68, 121.11, 120.49, 112.42, 109.82, 108.46, 108.31, 55.62, 43.94, 21.32. HRMS (ESI) Data for C₁₇H₁₆O₄[M+H]⁺ Calculated=285.1121. Found=285.1130. IR (Vmax) (Diamond ATR) cm⁻¹: 2992, 2834, 1686, 1598, 1492, 1465, 1241, 1165, 1188.

### Example 26

### (6) Synthesis of 1-(3-methoxyphenyl)-2-(4-methylbenzo[d][1,3]dioxol-2-yl)ethan-1-one

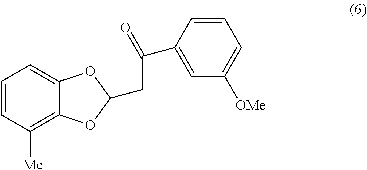 [SMILES: COc1cccc(C(=O)CC2Oc3cccc(C)c3O2)c1]

Title compound was synthesized using the general procedure B, starting from ynone (50 mg, 0.312 mmol, 1 eq.), K₂CO₃ (43.14 mg, 0.312 mmol, 1 eq.), acetone (4 ml) and catechol derivative (38.75 mg, 0.312 mmol). The desired compound was purified through column chromatography (20% EtOAc:Hexane) and isolated as sticky liquid in 87% (77.3 mg, 0.271 mmol) yield. R_{f}=0.4 (EtOAc:Hexane in 20:80). ¹H NMR (400 MHz, CDCl₃): δ 7.53 (ddd, J=7.6, 1.6, 1.2 Hz, 2H), 7.39 (dd, J=8.0, 7.6 Hz, 1H), 7.14 (ddd, J=8.2, 2.8, 1.2 Hz, 1H), 6.75-6.70 (m, 2H), 6.65 (d, J=7.2 Hz, 2H), 3.86 (s, 3H), 3.61 (d, J=5.6 Hz, 2H), 2.21 (s, 3H). ¹³C NMR (100 MHz, CDCl₃): δ 195.39, 160.05, 146.68, 145.56, 138.01, 129.85, 123.62, 121.40, 121.12, 120.45, 119.41, 112.41, 108.15, 106.40, 55.60, 44.09, 14.80. HRMS (ESI) Data for C₁₇H₁₆O₄ [M+H]⁺ Calculated=285.1121. Found=285.1130. IR(Vmax) (Diamond ATR) cm⁻¹: 3069, 2955, 2925, 1617, 1595, 1427.

### Example 27

### (7) Synthesis of 1-(3-methoxyphenyl)-2-(5-nitrobenzo[d][1,3]dioxol-2-yl)ethan-1-one

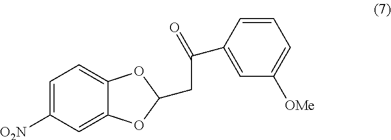 [SMILES: COc1cccc(C(=O)CC2Oc3ccc([N+](=O)[O-])cc3O2)c1]

The title compound was synthesized by following general procedure B, ynone (50 mg, 0.312 mmol, 1 eq.), K₂CO₃ (43.14 mg, 0.312 mmol, 1 eq.), acetone (4 ml) and catechol derivative (48.39 mg, 0.312 mmol, 1 eq.). The crude material was further purified through recrystallization in MeOH:DCM (4:1) and isolated as yellow solid in 90% (88.8 mg, 0.281 mmol) yield. R_{f}=0.5 (EtOAc:Hexane in 30:70). ¹H NMR (400 MHz, CDCl₃): δ 7.87 (dd, J=8.8, 2.4 Hz, 1H), 7.64-7.62 (m, 1H), 7.50 (dd, J=7.6, 7.6 Hz, 2H), 7.39 (ddd, J=8.4, 8.0, 2.0 Hz, 1H), 7.16-7.14 (m, 1H), 6.91 (d, J=5.2 Hz, 1H), 6.84 (ddd, J=8.8, 2.0, 1.6 Hz, 1H), 3.85 (s, 3H), 3.67 (d, J=5.2 Hz, 2H). ¹³C NMR (100 MHz, CDCl₃): δ 194.40, 160.14, 152.91, 147.98, 143.01, 137.53, 129.99, 120.95, 120.63, 120.01, 112.50, 111.39, 107.80, 104.69, 55.62, 43.69. HRMS (ESI) Data for C₁₆H₁₃NO₆ [M+H]⁺ Calculated=316.0816. Found=316.0830. IR(Vmax) (Diamond ATR) cm⁻¹: 3118, 3066, 2930, 1677, 1513, 1487, 1427. M. P.=82-84° C.

### Example 28

### (8) Synthesis of 1-(2-bromo-5-methoxyphenyl)-2-(5-methylbenzo [d][1,3]dioxol-2-yl)ethan-1-one

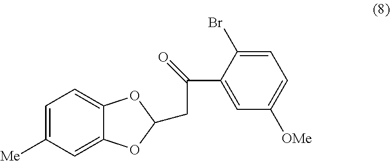 [SMILES: COc1ccc(Br)c(C(=O)CC2Oc3ccc(C)cc3O2)c1]

Title compound was synthesized using the general procedure B, starting from ynone (50 mg, 0.209 mmol, 1 eq.), K₂CO₃ (29 mg, 0.209 mmol, 1 eq.), DCM (4 ml) and catechol derivative (25.9 mg, 0.209 mmol, 1 eq.). The crude material was purified through column chromatography (15% EtOAc:Hexane) isolated as sticky liquid in 87% (65.9 mg, 0.181 mmol) yield. R_{f}=0.5 (EtOAc:Hexane in 20:80). ¹H NMR (400 MHz, CDCl₃): δ 7.47 (d, J=8.8 Hz, 1H), 6.99 (d, J=3.2 Hz, 1H), 6.87 (dd, J=8.8, 3.2 Hz, 1H), 6.67 (d, J=8.0 Hz, 1H), 6.63-6.61 (m, 3H), 3.80 (s, 3H), 3.63 (d, J=5.2 Hz, 2H), 2.26 (s, 3H). ¹³C NMR (100 MHz, CDCl₃): δ 198.87, 159.04, 144.98, 141.42, 134.78, 131.68, 121.69, 118.67, 114.14, 109.77, 109.27, 108.26, 108.26, 55.85, 47.74, 29.82, 21.29. HRMS (ESI) DATA for C₁₇H₁₅BrO₄ [M+H]⁺ Calculated=363.0226. Found=363.0236. IR (Vmax) (Diamond ATR) cm⁻¹: 2963, 2922, 1698, 1592, 1568, 1495, 690.

### Example 29

### (9c) Synthesis of 1-(2-bromo-5-methoxyphenyl)-2-(4-methoxybenzo [d][1,3]dioxol-2-yl)ethan-1-one

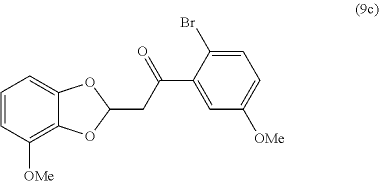 [SMILES: COc1ccc(Br)c(C(=O)CC2Oc3cccc(OC)c3O2)c1]

Title compound was synthesized using the general procedure B, starting from ynone (50 mg, 0.209 mmol, 1 eq.), DCM (4 ml), K₂CO₃ (29 mg, 0.209 mmol, 1 eq.) and catechol derivative (29.30 mg, 0.209 mmol, 1 eq.). The crude material was purified through column chromatography (15% EtOAc:Hexane) and isolated as sticky liquid in 89% (70.9 mg, 0.187 mmol) yield. R_{f}=0.4 (EtOAc:Hexane in 20:80). ¹H NMR (400 MHz, CDCl₃): δ 7.48 (d, J=8.8 Hz, 1H), 7.01 (d, J=3.2 Hz, 1H), 6.86 (dd, J=8.8, 2.8 Hz, 1H), 6.77 (dd, J=8.8, 0.8 Hz, 1H), 6.67 (dd, J=4.8, 4.8 Hz, 1H), 6.51 (m, 2H), 3.87 (s, 3H), 3.80 (s, 3H), 3.68 (dd, J=4.8, 2.8 Hz, 2H). ¹³C NMR (100 MHz, CDCl₃): δ 198.61, 159.05, 148.46, 144.06, 141.38, 134.96, 134.78, 122.20, 118.80, 114.45, 109.32, 108.69, 107.61, 102.60, 56.62, 55.80, 47.67. HRMS (ESI) DATA for C₁₇H₁₅BrO₅ [M+H]⁺ Calculated=379.0176. Found=379.0195. IR (Vmax)(Diamond ATR) cm⁻¹: 2960, 2909, 1702, 1592, 1568, 1495.

### Example 30

### (9a) Synthesis of 2-(4-methoxybenzo[d][1,3]dioxol-2-yl)-1-(2-nitrophenyl) ethan-1-one

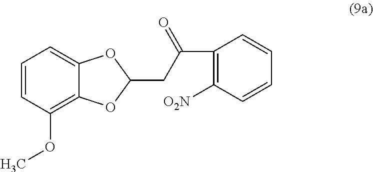 [SMILES: COc1cccc2c1OC(CC(=O)c1ccccc1[N+](=O)[O-])O2]

Title compound was synthesized using the general procedure B, starting from ynone (50 mg, 0.285 mmol, 1 eq.), 4 ml DCM, K2CO3 (1.96 mg, 5 mol %) and catechol derivative (39.93 mg, 0.285 mmol). The crude material was purified through column chromatography (10% EtOAc:Hexane) and isolated as white solid in 84% (75.5 mg, 0.239 mmol) yield. R_{f}=0.5 (EtOAc:Hexane in 20:80). ¹H NMR (400 MHz, CDCl₃): 8.10 (dd, J=8.0, 1.2 Hz, 1H), 7.71 (ddd, J=7.6, 1.2 Hz, 1H), 7.59 (dd, J=8.0, 1.6 Hz, 1H), 7.45 (dd, J=7.6, 1.6 Hz, 1H), 6.75 (dd, J=8.4, 8.4 Hz, 1H), 6.62 (t, J=5.2 Hz, 1H), 6.47 (dd, J=8.4, 1.2 Hz, 2H), 3.85 (s, 3H), 3.51 (dd, J=5.2, 2.0 Hz, 2H). ¹³C NMR (100 MHz, CDCl₃): δ 197.51, 148.26, 145.55, 144.02, 137.26, 134.53, 130.98, 127.73, 124.40, 122.23, 108.13, 107.72, 102.52, 67.13, 56.58, 47.53. HRMS DATA for C₁₆H₁₃NO₆ [M+H]⁺ Calculated=316.0816. Found=316.0829. IR (Vmax) (Diamond ATR) cm⁻¹: 2916, 1705, 1638, 1601, 1284, 1178, 1091. M. P.=105-107° C.

### Example 31

### (9b) Synthesis of 2-(benzo[d][1,3]dioxol-2-yl)-1-(3-methoxyphenyl)ethan-1-one

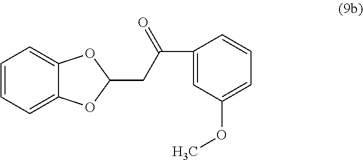 [SMILES: COc1cccc(C(=O)CC2Oc3ccccc3O2)c1]

Title compound was synthesized using the general procedure B, starting from ynone (50 mg, 0.312 mmol, 1 q.), 4 ml acetone, K2CO3 (2.15 mg, 5 mol %) and benzene-1,2-diol (34.37 mg, 0.312 mmol). The crude material was purified through column chromatography (10% EtOAc:Hexane) and isolated as white solid in 89% (75.2 mg, 0.278 mmol) yield. R_{f}=0.5 (EtOAc:Hexane in 20:80). ¹H NMR (400 MHz, CDCl₃): 7.54-7.50 (m, 2H), 7.39 (dd, J=8.0, 8.0 Hz, 1H), 7.15 (ddd, J=8.0, 2.4, 1.2 Hz, 1H), 6.82 (s, 4H), 6.73 (t, J=5.2 Hz, 1H), 3.86 (s, 3H), 3.62 (d, J=5.2 Hz, 2H). ¹³C NMR (100 MHz, CDCl₃): δ 195.35, 160.07, 147.22, 137.94, 137.94, 129.88, 121.81, 121.81, 121.08, 120.49, 112.43, 108.92, 108.92, 108.42, 55.61, 43.95. HRMS (ESI) DATA for C₁₆H₁₄O₄[M+H⁺] Calculated=271.0965. Found=271.0970. IR(Vmax) (Diamond ATR) cm⁻¹: 2918, 1685, 1578, 1236, 1115, 1047. M. P.=102-103° C.

### Example 32

### (10) Synthesis of 2-(benzo[d][1,3]oxathiol-2-yl)-1-(3-methoxyphenyl) ethan-1-one

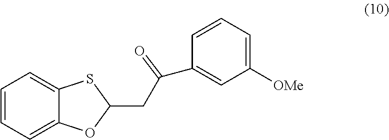 [SMILES: COc1cccc(C(=O)CC2Oc3ccccc3S2)c1]

Title compound was synthesized using the general procedure B, starting from ynone (50 mg, 0.312 mmol, 1 eq.), DCM (4 ml), K2CO3 (2.15 mg, 5 mol %) and 2-mercaptophenol (39.36 mg, 0.312 mmol). The crude material was purified through column chromatography (20% EtOAc:Hexane) and isolated as pale white solid in 91% (81.5 mg, 0.284 mmol) yield. R_{f}=0.5 (EtOAc:Hexane in 20:80). ¹H NMR (400 MHz, CDCl3): δ 7.52-7.48 (m, 2H), 7.41-7.34 (m, 1H), 7.15-7.12 (m, 2H), 6.99 (dq, J=8.0, 1.6, 1.2 Hz, 1H), 6.88 (td, J=7.6, 1.2 Hz, 1H), 6.81 (dd, J=8.0, 1.2 Hz, 1H), 6.56 (dd, J=7.2, 5.2 Hz, 1H), 3.87 (dd, J=17.6, 5.2 Hz, 1H), 3.88 (s, 3H), 3.64 (dd, J=17.6, 7.2 Hz, 1H). ¹³C NMR (100 MHz, CDCl3): δ 196.35, 160.06, 155.23, 137.67, 129.87, 125.93, 125.64, 122.55, 122.21, 120.97, 120.50, 112.28, 110.61, 85.66, 55.60, 46.71. HRMS (ESI) DATA for C₁₆H₁₄O₃S [M+H]⁺ Calculated=287.0736. Found=287.0744. IR(Vmax) (Diamond ATR) cm⁻¹: 2913, 2837, 1671, 1595, 1247, 1159, 1091. M. P.=76-78° C.

### Example 33

### (11) Synthesis of 2-(benzo[d][1,3]dithiol-2-yl)-1-(2-nitrophenyl)ethan-1-one

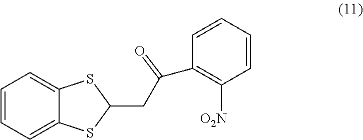 [SMILES: O=C(CC1Sc2ccccc2S1)c1ccccc1[N+](=O)[O-]]

Title compound was synthesized using the general procedure B, starting from ynone (50 mg, 0.285 mmol, 1 q.), DCM (4 ml), K2CO3 (1.96 mg, 5 mol %) and benzene-1,2-dithiol (40.60 mg, 0.285 mmol). The crude material was purified through column chromatography (10% EtOAc:Hexane) and isolated as white solid in 92% (83 mg, 0.261 mmol) yield. R_{f}=0.5 (EtOAc:Hexane in 20:80). ¹H NMR (400 MHz, CDCl3): δ 8.10 (dd, J=8.0, 1.2 Hz, 1H), 7.71 (ddd, J=15.2, 7.6, 1.2 Hz, 1H), 7.62-7.58 (m, 1H), 7.41 (dd, J=7.2, 1.2 Hz, 1H), 7.24-7.22 (m, 2H), 7.04-7.02 (m, 2H), 5.33 (t, J=7.2 Hz, 1H), 3.52 (d, J=7.2 Hz, 2H). ¹³C NMR (100 MHz, CDCl₃): δ 190.15, 145.69, 137.11, 136.72, 134.45, 134.45, 131.01, 127.63, 125.83, 125.83, 124.62, 122.80, 122.80, 52.42, 47.71. HRMS (ESI) DATA for C₁₅H₁₁NO₃S₂[M+H]⁺ Calculated=317.02. Found=317.0289. IR (Vmax) (Diamond ATR) cm⁻¹: 2931, 1686, 1526, 1342, 1216. M. P.=129-131° C.

### Example 34

### (12) Synthesis of 2-(benzo[d][1,3]dithiol-2-yl)-1-(3-methoxyphenyl) ethan-1-one

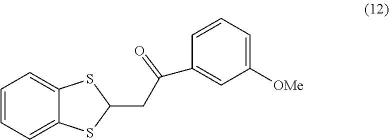 [SMILES: COc1cccc(C(=O)CC2Sc3ccccc3S2)c1]

Title compound was synthesized using the general procedure B, starting from ynone (50 mg, 0.312 mmol, 1 eq.), DCM (4 ml), K2CO3 (2.15 mg, 5 mol %) and benzene-1,2-dithiol (44.37 mg, 0.312 mmol, 1 eq.). The crude material was purified through column chromatography (15% EtOAc:Hexane) and isolated as white solid in 86% (80.8 mg, 0.267 mmol) yield. R_{f}=0.5 (EtOAc:Hexane in 20:80). ¹H NMR (400 MHz, CDCl₃): δ 7.48-7.45 (m, 2H), 7.36-7.31 (m, 1H), 7.24-7.20 (m, 2H), 7.11 (ddd, J=8.4, 2.8, 1.2 Hz, 1H), 7.06-7.02 (m, 2H), 5.34 (t, J=7.2 Hz, 1H), 3.84 (s, 3H), 3.66 (d, J=7.2 Hz, 2H). ¹³C NMR (100 MHz, CDCl₃): δ 196.67, 160.00, 137.60, 136.98, 129.81, 129.81, 125.71, 125.71, 122.77, 122.77, 120.87, 120.39, 112.23, 55.60, 48.92, 48.19. HRMS (ESI) DATA for C₁₆H₁₄O₂S₂ [M+H]⁺ Calculated=303.0508. Found=303.0346. IR(Vmax) (Diamond ATR) cm⁻¹: 2938, 1666, 1580, 1249, 1201, 1118, 1042. M. P.=98-100° C.

### Example 35

### (13) Synthesis of 2-(dinaphtho[2,1-d:1′,2′-f][1,3]dioxepin-4-yl)-1-(3 methoxyphenyl)ethan-1-one

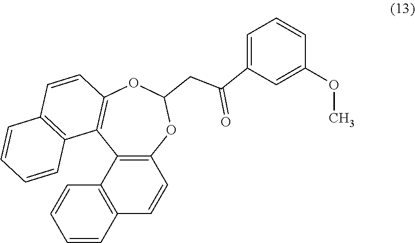 [SMILES: COc1cccc(C(=O)CC2Oc3ccc4ccccc4c3-c3c(ccc4ccccc34)O2)c1]

The title compound was synthesized by following general procedure B, ketone (50 mg, 0.312 mmol, 1 eq.), 4 ml THF, K2CO3 (2.15 mg, 5 mol %) and catechol (89.33 mg, 0.312 mmol, 1 eq.). The crude material was purified by (20% EtOAc:Hexane) and isolated as off white solid powder in 94% (131.2 mg, 0.293 mmol) yield. R_{f}=0.5 (EtOAc:Hexane in 20:80). ¹H NMR (400 MHz, CDCl₃): δ 7.99-7.93 (m, 4H), 7.54-7.43 (m, 7H), 7.40-7.28 (m, 4H), 7.13 (ddd, J=8.4, 2.4, 1.2 Hz, 1H), 6.44 (t, J=5.2 Hz, 1H), 3.84 (s, 3H), 3.54 (d, J=5.2 Hz, 2H). ¹³C NMR (100 MHz, CDCl₃): δ 195.65, 160.07, 151.85, 149.06, 138.31, 132.45, 132.24, 132.06, 131.78, 130.50, 129.90, 129.87, 128.56, 128.45, 127.14, 127.11, 126.22, 126.20, 126.11, 126.09, 125.22, 125.18, 122.93, 121.15, 121.11, 120.32, 112.49, 109.92, 55.59, 42.80. HRMS (ESI) Data for C₃₀H₂₂O₄[M+H]⁺ Calculated=447.1552. Found=447.1607. IR(Vmax) (Diamond ATR) cm⁻¹: 2975, 2906, 1736, 1680, 1153, 1175, 1120, 1029. M. P.=143-145° C.

In one of the embodiment of the present invention the process for synthesis of novel co-initiator for photo induced radical polymerization represented by substituted [1,3]-benzodioxole, [1,3]-benzodithianes, [1,3]-benzothioxole, [1, 3]-benzoxathiin-4-ones and [1,3]dioxepin derivative is attained by the above mentioned process.

In one of the embodiment of the present invention the process for synthesis of novel co-initiator for photo induced radical polymerization, the base is selected from potassium carbonate, sodium carbonate, sodium hydroxide, sodium acetate, potassium tertiary butoxide, sodium tertiarybutoxide, barium hydroxide, sodium amide and potassium carbonate turned to be the best catalyst.

In one of the embodiments of the present invention, the co-initiator for photo induced radical polymerization, the solvent is selected from dichloromethane, methanol, acetonitrile, toluene, tetrahydrofuran, and dichloromethane seems to be the best solvent.

In one of the embodiments of the present invention, the compounds of Formula 3, Formula 5, Formula C and Formula Z are selected from:
1. 2-(2-(3-methoxyphenyl)-2-oxoethyl)-4H-benzo[d][1,3]oxathiin-4-one (2a)
2. 2-(2-(3,4-dimethoxyphenyl)-2-oxoethyl)-4H-benzo[d][1,3]oxathiin-4-one (2b)
3. 2-(2-oxo-2-(p-tolyl)ethyl)-4H-benzo[d][1,3]oxathiin-4-one (2c)
4. 4-(2-(4-oxo-4H-benzo[d][1,3]oxathiin-2-yl)acetyl)benzonitrile (2d)
5. 2-(2-(4-methoxyphenyl)-2-oxoethyl)-4H-benzo[d][1,3]oxathiin-4-one (2e)
6. 2-(2-oxo-2-(3,4,5-trimethoxyphenyl)ethyl)-4H-benzo[d][1,3]oxathiin-4-one (2f)
7. 2-(2-oxo-2-phenylethyl)-4H-benzo[d][1,3]oxathiin-4-one (2g)
8. 2-(2-(4-chlorophenyl)-2-oxoethyl)-4H-benzo[d][1,3]oxathiin-4-one (2h)
9. 2-(2-(4-fluorophenyl)-2-oxoethyl)-4H-benzo[d][1,3]oxathiin-4-one (2i)
10. 2-(2-(4-bromophenyl)-2-oxoethyl)-4H-benzo[d][1,3]oxathiin-4-one (2j)
11. 2-(2-(4-nitrophenyl)-2-oxoethyl)-4H-benzo[d][1,3]oxathiin-4-one (2k)
12. 2-(2-(4-(allyloxy)phenyl)-2-oxoethyl)-4H-benzo[d][1,3]oxathiin-4-one (2l)
13. 2-(2-oxo-2-(3-phenoxyphenyl)ethyl)-4H-benzo[d][1,3]oxathiin-4-one (2m)
14. 2-(2-(6-bromobenzo[d][1,3]dioxol-5-yl)-2-oxoethyl)4Hbenzo[d][1,3]oxathiin-4-one (2n)
15. 2-(2-(2-bromo-5-methoxyphenyl)-2-oxoethyl)-4H-benzo[d][1,3]oxathiin-4-one (2o)
16. 4-(2-(4-oxo-4H-benzo[d][1,3]oxathiin-2-yl)acetyl)phenyl4-methyl benzenesulfonate (2p)
17. 2-(2-cyclopropyl-2-oxoethyl)-4H-benzo[d][1,3]oxathiin-4-one (2q)
18. 2-(2-oxo-4-phenylbutyl)-4H-benzo[d][1,3]oxathiin-4-one (2r)
19. 2-(2-(cyclohex-2-en-1-yl)-2-oxoethyl)-4H-benzo[d][1,3]oxathiin-4-one (2s)
20. 2-(2-(2-bromo-5-methoxyphenyl)-2-oxoethyl)-4H-benzo[d][1, 3]oxathiin-4-one (2t)
21. 2-(5-(tert-butyl)benzo[d][1,3]dioxol-2-yl)-1-(3-methoxyphenyl)ethan-1-one (1)
22. 2-(4,6-di-tert-butyl benzo[d][1,3]dioxol-2-yl)-1-(3 methoxyphenyl)ethan-1-one (2)
23. 2-(4-methoxybenzo[d][1,3]dioxol-2-yl)-1-(3-methoxyphenyl)ethan-1-one (3)
24. 2-(2-(3-methoxyphenyl)-2-oxoethyl) benzo[d][1,3]dioxole-5-carbonitrile (4)
25. 1-(3-methoxyphenyl)-2-(5-methylbenzo[d][1,3]dioxol-2-yl)ethan-1-one (5)
26. 1-(3-methoxyphenyl)-2-(4-methylbenzo[d][1,3]dioxol-2-yl)ethan-1-one (6)
27. 1-(3-methoxyphenyl)-2-(5-nitrobenzo[d][1,3]dioxol-2-yl)ethan-1-one (7)
28. 1-(2-bromo-5-methoxyphenyl)-2-(5-methylbenzo[d][1,3]dioxol-2-yl)ethan-1-one (8)
29. 1-(2-bromo-5-methoxyphenyl)-2-(4-methoxybenzo[d][1,3]dioxol-2-yl)ethan-1-one (9c)
30. 2-(4-methoxybenzo[d][1,3]dioxol-2-yl)-1-(2-nitrophenyl) ethan-1-one (9a)
31. 2-(benzo[d][1,3]dioxol-2-yl)-1-(3-methoxyphenyl)ethan-1-one (9b)
32. 2-(benzo[d][1,3]oxathiol-2-yl)-1-(3-methoxyphenyl) ethan-1-one (10)
33. 2-(benzo[d][1,3]dithiol-2-yl)-1-(2-nitrophenyl)ethan-1-one (11)
34. 2-(benzo[d][1,3]dithiol-2-yl)-1-(3-methoxyphenyl) ethan-1-one (12)
35. 2-(dinaphtho[2,1-d:1′,2′-f][1,3]dioxepin-4-yl)-1-(3 methoxyphenyl)ethan-1-one (13)

### Advantages of the Present Invention

The process as disclosed in the present invention has tremendous advantages over any of the processes disclosed for similar reactions hitherto.
a. operational simplicity.
b. high atom economy.
c. ease of purification and bench stable.
d. scalable with very high level of functional group tolerance.
e. cheap commercially available reactant.
f. Reaction is performed at room temperature.

Thus, the present invention discloses an innovative and highly atom economical process for synthesis of novel co-initiator for photo-induced radical polymerization technique. Moreover, the developed invention is a composition with multiple functionalities. Therefore, new co-initiator of the present invention having 6,5 and 6,6-bicyclic scaffolds A & B as central core makes the present invention more efficient, resourceful and economic.

Although the invention has now been described in terms of certain preferred embodiments and exemplified with respect thereto, one skilled in art can readily appreciate that various modifications, changes, omissions and substitutions may be made without departing from the spirit thereof. It is intended therefore that the present invention be limited solely by the scope of the following claims.

We claim:

1. A process of preparation of co-initiator for photo induced radical polymerization, comprising:
i. reacting compound of Formula 1 with compound of formula 2, in the presence of a base at room temperature yields compound of formula 3; 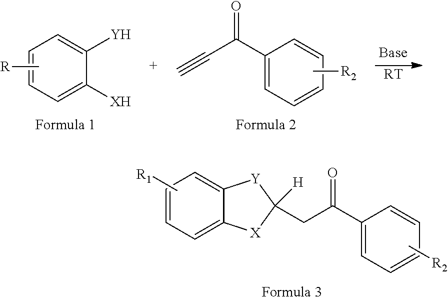
ii. reacting compound of Formula 1 with compound of formula 4, in presence of a base at room temperature yields compound of formula 5; 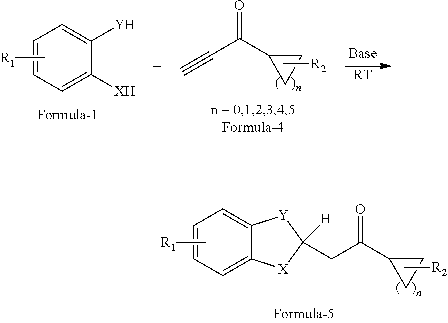
wherein,
X is selected from the group comprising of O, S, N, Se and Te;
Y is selected from the group comprising of O, S, N, Se and Te;
wherein R₁ is selected from the group consisting Hydro, alkyl, branched chain alkyl, aryl, t-butyl, methyl, cyano, o-methyl, Nitro and also comprises tert-Butyloxycarbonyl (BocHN); and
various acyclic and cyclic ketones of formula 4 are selected from n=0, 1, 2, 3, 4, and R₂ is selected from group comprising of OMe, Me, CN, Methyl, Cl, F, Br, NO₂, OPh, OTs, allylic and the like;
iii. reacting compound of Formula A with compound of formula 2 (ketone), in the presence of a base at room temperature yields compound of formula C; 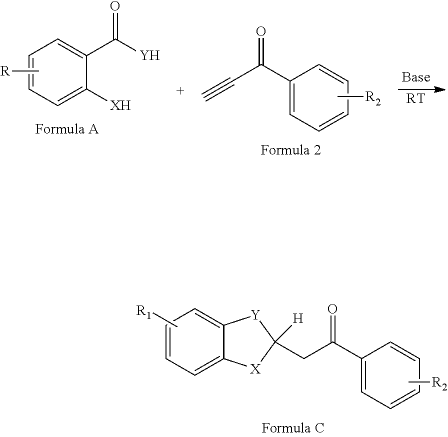
wherein, formula C
X is selected from O, S, N, Se and Te;
Y is selected from O, S, N, Se and Te;
Wherein R₁ is selected from the group consisting Hydro, alkyl, branched chain alkyl, aryl, t-butyl, methyl, cyano, o-methyl, Nitro and also comprises tert-Butyloxycarbonyl (BocHN); and
R₂ is selected from R₂ is selected from group comprising of OMe, Me, CN, Methyl, Cl, F, Br, NO₂, OPh, OTs, allylic and the like.
iv. reacting compound of Formula X with compound of formula 2, in the presence of a base at room temperature yields compound of formula Z 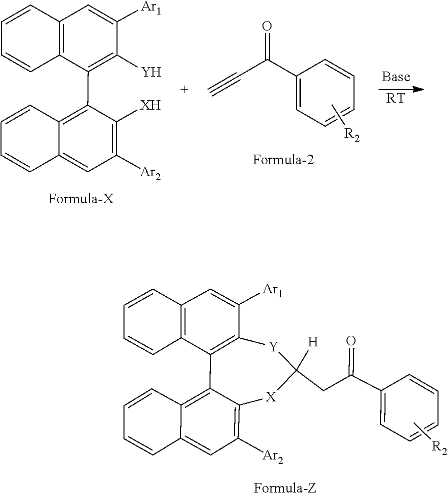
wherein in Formula Z
X=O, N, S
Y=O, N, S
Ar₁=p-CF₃—C₆H₅, naphthyl
Ar₂=p-CF₃—C₆H₅, naphthyl
wherein, R₂ is selected from group comprising of OMe, Me, CN, Methyl, Cl, F, Br, NO₂, OPh, OTs, allylic and the like.

2. The process of preparation of co-initiator for photo induced radical polymerization as claimed in claim 1, wherein the ratio of compound of Formula A, Formula 1 and Formula X with Formula 2 is in the ratio of 1:1 equivalents and Formula 1 with Formula 4 is in a ratio of 1:1 equivalents.

3. The process of preparation of co-initiator for photo induced radical polymerization as claimed in claim 1, wherein the base is selected from Potassium Carbonate, Sodium carbonate, sodium hydroxide, sodium acetate, potassium tertiary butoxide, sodium tertiarybutoxide, barium hydroxide, sodium amide in a concentration range of 2-10 mol %.

4. The process of preparation of co-initiator for photo induced radical polymerization as claimed in claim 1, wherein the solvent is selected from polar and non-polar solvents such as dichloromethane, methanol, acetonitrile, toluene, tetrahydrofuran in a concentration range of 2-10 ml.

5. The process of preparation of co-initiator for photo induced radical polymerization as claimed in claim 1, wherein the reaction time to attain compound of Formula-3 is 5.5 hrs.

6. The process of preparation of co-initiator for photo induced radical polymerization as claimed in claim 1, wherein the reaction time to attain compound of Formula-C is 5 mins.

7. The process of preparation of co-initiator for photo induced radical polymerization as claimed in claim 1, wherein the reaction time to attain compound of Formula-5 is up to 5.5 hrs.

8. The process of preparation of co-initiator for Photo induced Radical Polymerization as claimed in claim 1, wherein the reaction time to attain compound of Formula-Z is 5.5 hrs.

9. The process of preparation of co-initiator for Photo induced Radical Polymerization as claimed in claim 1, wherein in the Formula 3 compound comprises. 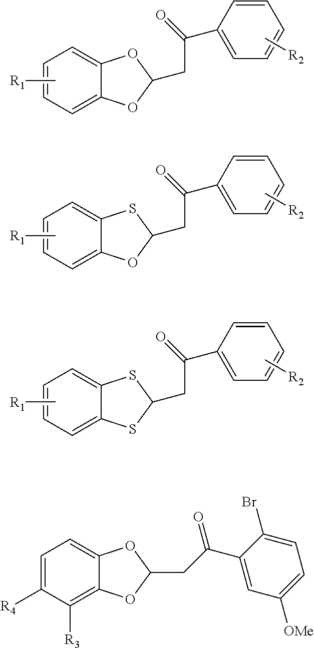
in which
R₁ is selected from group comprising of Hydro, alkyl branched chain alkyl, aryl, t-butyl, methyl, cyano, o-methyl, Nitro and also comprises tert-Butyloxycarbonyl (BocHN);
R₂ is selected from group comprising of OMe, Me, CN, Methyl, Cl, F, Br, NO₂, OPh, OTs, allylic and the like,
R₃ is selected from the group comprising of Me, H, and
R₄ is selected from group comprising of Me, H.

10. The process of preparation of co-initiator for photo induced radical polymerization as claimed in claim 1, wherein in the formula-C is the compound comprising: 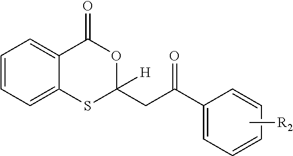
wherein, R₂ is selected from the group comprising of OMe, Me, CN, Methyl, Cl, F, Br, NO₂, OPh, OTs, allylic and the like.

11. The process of preparation of co-initiator for photo induced radical polymerization as claimed in claim 1, wherein the compounds of Formula 3, Formula 5, Formula C and Formula Z are selected from:
1. 2-(2-(3-methoxyphenyl)-2-oxoethyl)-4H-benzo[d][1,3]oxathiin-4-one (2a)
2. 2-(2-(3,4-dimethoxyphenyl)-2-oxoethyl)-4H-benzo[d][1,3]oxathiin-4-one (2b)
3. 2-(2-oxo-2-(p-tolyl)ethyl)-4H-benzo[d][1,3]oxathiin-4-one (2c)
4. 4-(2-(4-oxo-4H-benzo[d][1,3]oxathiin-2-yl)acetyl)benzonitrile (2d)
5. 2-(2-(4-methoxyphenyl)-2-oxoethyl)-4H-benzo[d][1,3]oxathiin-4-one (2e)
6. 2-(2-oxo-2-(3,4,5-trimethoxyphenyl)ethyl)-4H-benzo[d][1,3]oxathiin-4-one (2f)
7. 2-(2-oxo-2-phenylethyl)-4H-benzo[d][1,3]oxathiin-4-one (2g)
8. 2-(2-(4-chlorophenyl)-2-oxoethyl)-4H-benzo[d][1,3]oxathiin-4-one (2h)
9. 2-(2-(4-fluorophenyl)-2-oxoethyl)-4H-benzo[d][1,3]oxathiin-4-one (2i)
10. 2-(2-(4-bromophenyl)-2-oxoethyl)-4H-benzo[d][1,3]oxathiin-4-one (2j)
11. 2-(2-(4-nitrophenyl)-2-oxoethyl)-4H-benzo[d][1,3]oxathiin-4-one (2k)
12. 2-(2-(4-(allyloxy)phenyl)-2-oxoethyl)-4H-benzo[d][1,3]oxathiin-4-one (2l)
13. 2-(2-oxo-2-(3-phenoxyphenyl)ethyl)-4H-benzo[d][1,3]oxathiin-4-one (2m)
14. 2-(2-(6-bromobenzo[d][1,3]dioxol-5-yl)-2-oxoethyl)4Hbenzo[d][1,3]oxathiin-4-one (2n)
15. 2-(2-(2-bromo-5-methoxyphenyl)-2-oxoethyl)-4H-benzo[d][1,3]oxathiin-4-one (2o)
16. 4-(2-(4-oxo-4H-benzo[d][1,3]oxathiin-2-yl)acetyl)phenyl4-methyl benzenesulfonate (2p)
17. 2-(2-cyclopropyl-2-oxoethyl)-4H-benzo[d][1,3]oxathiin-4-one (2q)
18. 2-(2-oxo-4-phenylbutyl)-4H-benzo[d][1,3]oxathiin-4-one (2r)
19. 2-(2-(cyclohex-2-en-1-yl)-2-oxoethyl)-4H-benzo[d][1,3]oxathiin-4-one (2s)
20. 2-(2-(2-bromo-5-methoxyphenyl)-2-oxoethyl)-4H-benzo[d][1, 3]oxathiin-4-one (2t)
21. 2-(5-(tert-butyl)benzo[d][1,3]dioxol-2-yl)-1-(3-methoxyphenyl)ethan-1-one (1)
22. 2-(4,6-di-tert-butyl benzo[d][1,3]dioxol-2-yl)-1-(3 methoxyphenyl)ethan-1-one (2)
23. 2-(4-methoxybenzo[d][1,3]dioxol-2-yl)-1-(3-methoxyphenyl)ethan-1-one (3)
24. 2-(2-(3-methoxyphenyl)-2-oxoethyl) benzo[d][1,3]dioxole-5-carbonitrile (4)
25. 1-(3-methoxyphenyl)-2-(5-methylbenzo[d][1,3]dioxol-2-yl)ethan-1-one (5)
26. 1-(3-methoxyphenyl)-2-(4-methylbenzo[d][1,3]dioxol-2-yl)ethan-1-one (6)
27. 1-(3-methoxyphenyl)-2-(5-nitrobenzo[d][1,3]dioxol-2-yl)ethan-1-one (7)
28. 1-(2-bromo-5-methoxyphenyl)-2-(5-methylbenzo[d][1,3]dioxol-2-yl)ethan-1-one (8)
29. 1-(2-bromo-5-methoxyphenyl)-2-(4-methoxybenzo[d][1,3]dioxol-2-yl)ethan-1-one (9c)
30. 2-(4-methoxybenzo[d][1,3]dioxol-2-yl)-1-(2-nitrophenyl) ethan-1-one (9a)
31. 2-(benzo[d][1,3]dioxol-2-yl)-1-(3-methoxyphenyl)ethan-1-one (9b)
32. 2-(benzo[d][1,3]oxathiol-2-yl)-1-(3-methoxyphenyl) ethan-1-one (10)
33. 2-(benzo[d][1,3]dithiol-2-yl)-1-(2-nitrophenyl)ethan-1-one (11)
34. 2-(benzo[d][1,3]dithiol-2-yl)-1-(3-methoxyphenyl) ethan-1-one (12)
35. 2-(dinaphtho[2,1-d:1′,2′-f][1,3]dioxepin-4-yl)-1-(3 methoxyphenyl)ethan-1-one (13)
